(19) United States
(12) Patent Application Publication
(10) Pub. No.: US 2025/0110116 A1
(43) Pub. Date: Apr. 3, 2025

(54) COMPOSITIONS AND METHODS FOR PRODUCING HAIR FOLLICLE STEM CELLS

(71) Applicant: The Rockefeller University, New York, NY (US)

(72) Inventors: Elaine Fuchs, New York, NY (US); Matthew Tierney, New York, NY (US)

(21) Appl. No.: 18/902,455

(22) Filed: Sep. 30, 2024

Related U.S. Application Data
(60) Provisional application No. 63/586,521, filed on Sep. 29, 2023.

(51) Int. Cl. G01N 33/50; C12N 5/071

(52) U.S. Cl. CPC G01N 33/5044; C12N 5/0627; C12N 2501/01; C12N 2501/11; C12N 2501/119; C12N 2501/727

(57) ABSTRACT

Provided are compositions and methods that enable faithful recapitulation of hair follicle stem cells (HFSCs) identity in culture using a cocktail of factors that include use of an inhibitor of protein kinase C (PKCi), and all-trans retinoic acid (atRA), that successfully target lineage plasticity.

### CROSS-REFERENCE TO RELATED APPLICATION

This application claims the benefit of U.S. provisional application No. 63/586,521, filed Sep. 29, 2023, the entire disclosure of which is incorporated herein by reference.

### STATEMENT REGARDING FEDERALLY SPONSORED RESEARCH

This invention was made with government support under grant nos. R01-AR31737, F32-AR073105, K99-AR079575 awarded by the National Institute of Arthritis Musculoskeletal and Skin Diseases (NIAMS). The government has certain rights in the invention.

### RELATED INFORMATION

The skin is a pioneering model for stem cell culture, beginning with seminal work describing the cultivation of epidermal stem cells on irradiated fibroblasts. Successive improvements, including the incorporation of cellularized dermal equivalents, introduction of epidermal growth factors, and late-stage exposure to an air-liquid interface, progressively enhanced their expansion and upward differentiation into stratified epithelia. These methods marked the first reconstitution of a three-dimensional (3D) structure from stem cells outside the body and quickly reached clinical relevance for autologous grafting in severe burn patients. Today, skin grafting techniques have FDA approval and are used in combination with gene therapy to regenerate the epidermis and restore the skin's primary function as a barrier from external pathogens for patients suffering from structural fragility, chronic blistering, or trauma.

In addition to its barrier function, the complete integumentary system of the skin includes the pilosebaceous unit, an appendage composed of the sebaceous gland and hair follicle. Extending inward from the epidermis, this dynamic mini-organ performs secretory and thermoregulatory duties. The follicular unit also aids in sensory reception by acting as an anchor for neurons and arrector pili muscles while providing a niche for melanocytes to contribute hair pigmentation and protect against ultraviolet (UV)-induced DNA damage. Thus, hair formation is essential to the production of fully functional skin grafts. However, de novo follicular neogenesis does not occur during the clinical production of grafted skin.

Hair formation and growth is fueled by long-lived hair follicle stem cells (HFSCs), which reside in a niche anatomically defined as the hair ‘bulge’ and constitute the cellular engine for cyclical bouts of regeneration. Maintaining the defining features of HFSCs in culture, including their self-renewal and differentiation to all cell fates of the hair follicle, remains a significant obstacle to regenerative medicine applications targeting hair biology, despite a wealth of knowledge describing many of the niche signals that control their behavior. Indeed, HFSCs purified by fluorescence activated cell sorting (FACS) and cultured using conventional methods can be differentiated to epidermal, but not hair follicle lineages and lose the expression of key transcription factors that govern processes essential to physiological HFSC behavior.

Early attempts to model HFSC behavior ex vivo primarily relied on explant systems in skin biopsies or micro-dissected hair follicles that only temporarily maintained cell viability. Influenced by the fibroblast ‘feeder cell’ layer in epidermal cultures and a growing appreciation for the role played by a condensed mesenchyme, termed the dermal papillae, in controlling HFSC activity, researchers have incorporated dermal mesenchymal cells into different culture systems. Recent attempts have shown improved outcomes, in some cases spontaneously forming sporadic hair-like structures over time. Simply reproducing the 3D structure of epidermal-mesenchymal interactions using 3D-printed scaffolds also produced differentiated cell fates in patterns that resembled hair follicles. However, these methods rely on the use of stem cells isolated from embryonic or early neonatal timepoints that quickly lose their ability to produce hair cell fates in these settings, and therefore cannot be employed in most clinical settings.

The indefinite propagation of adult HFSCs without niche cell types in culture has been accomplished using 3D hydrogels composed of various mixtures of extracellular matrix networks to support clonal outgrowth, building upon the recent developments of ‘organoid’ technology. However, while the expression of a subset of HFSC markers has been restored in this setting through the addition of factors that prevent anoikis and promote proliferation, these conditions do not fully restore HFSC identity or behavior. Efforts to activate pathways normally regulated by the niche in this context, including WNT, BMP, and TGFb signaling, have not resulted in the expected cell behaviors but rather produced inward-stratifying epidermal structures more like the skin than hair follicle. There thus an ongoing and unmet need to resolve factors that relate to hair follicle stem cell generation, and for compositions and methods that can be used to produce hair follicle stem cells and modulate their differentiation. The present disclosure is pertinent to this need.

### BRIEF SUMMARY

This disclosure provides a novel methodology that enables faithful recapitulation of hair follicle stem cells (HFSCs) identity in culture. In an example, this is achieved by using a cocktail of factors, including an inhibitor of protein kinase C (PKCi) and all-trans retinoic acid (atRA), that successfully target lineage plasticity. This molecularly defined state allows for stem cells to transition from one committed developmental lineage to another. By providing these factors, lineage plasticity is resolved and hair follicle stem cells are restored to their original, in vivo state. The disclosure also provides mixtures of epithelial cells and HFSCs, and methods for controlled growth of hair follicles

The disclosure provides a series of conditions using these and other factors that generate a suite of hair cell types, confirming the efficacy of the factors for restoring their inherent capacity for hair regeneration. The described platform overcomes a significant hurdle to modeling hair follicle stem cell behavior in a high-throughput and tractable manner, which has shown promise for disease-oriented and regenerative applications across a range of tissues. The described compositions and methods are expected to facilitate the rapid testing of both existing and new therapeutic modalities designed to treat hair and skin disorders, including cancer when lineage plasticity is high. The disclosure includes patient-specific preclinical testing of samples from individuals to provide safer treatments and significantly lower previously existing costs.

The disclosure includes data demonstrating targeting of retinoic acid signaling in vivo to rejuvenate stem cell behavior during wound repair in mouse and human cells. The disclosure includes leveraging aspect of the invention to overcome longstanding barriers to chronic wound healing deficits and limited follicular neogenesis in the context of burns or grafted skin, helping restore tissue functionality by directing stem cells to barrier reformation or endogenous appendage formation for specific needs that will be recognized by those skilled in the art when given the benefit of the present disclosure. Thus, the disclosure provides various approaches to regulating lineage plasticity and fate of hair follicle stem cells when cultured or engaged in wound repair processes and demonstrates, without intending to be bound by any particular theory, that retinoic acid dictates whether and when bipotent adult stem cells make one tissue versus another. The cell culture medium composition, and cells that are present in the cell culture medium, are also provided.

### BRIEF DESCRIPTION OF FIGURES

The patent or application file contains at least one drawing executed in color. Copies of th is patent or patent application publication with color drawing(s) will be provided by the Office upon request and payment of the necessary fee.

FIG. 1: Resolution of lineage plasticity in HFSCs ex vivo. (Panel A) Cultured HFSCs were stably transduced with a lentiviral Klf5-EGFP reporter (left). When grown in 3D cultures, flow cytometry quantification of Klf5-EGFP⁺ cells (center) showed that serum replacement reduced Klf5-EGFP levels. Cultured HFSC colony diameter plateaued over time in serum replacement media, in contrast to unrestrained outgrowth in serum-replete media (right; n=5). (Panel B) Quantification of Klf5-EGFP⁺ cells after screening a library of small molecules implicated in wound repair identified several small molecule ‘hits’ (n=5). The dashed line indicates baseline Klf5-EGFP levels in B-27 media with vehicle-only controls. (Panels C, D) Immunofluorescence (IF) and western blot of HFSC cultures following serum replacement and treatment with RA and PKCi (n=6) revealed alterations in SOX9-KLF5 expression and keratin profiles that reflected conditions permissive for epidermal differentiation and restoration of HFSC identity, respectively. The epidermis-dermis interface is outlined. Note that all cell compartments are labeled as follows: epi=epidermis, sg=sweat gland, dp=dermal papillae. Scale bar, 20 μm. (Panel E) UMAP representation and unsupervised k-nearest-neighbor-based clustering of single cell transcriptomes from cultured HFSCs (3,757 cells) after single cell RNA-sequencing (scRNA-seq) demonstrated that RA-PKCi co-treatment grouped cells in near-uniform clusters distinct from wound-like and epidermal cell types (left, center). Annotated cluster assignments established using gene signatures derived from independent bulk sequencing of HFSCs, EpSCs, and lineage traced HFSCs isolated at intermediate stages of wound repair confirmed that RA- and PKCi-treated cells possessed a strong HFSC signature and reduced EpSC and wound signatures (right).

FIG. 2: RA influences the balance between HFSC quiescence and activation. (Panel A) High levels of BMP activity in SOX9⁺ quiescent bulge HFSCs in vivo. The epidermis-dermis interface is outlined; asterisk refers to autofluorescence in the sweat gland. Scale bar, 50 μm. (Panel B) Quiescent HFSC marker Nfatc1 (left) and HFSC-associated Cxcl14-EGFP super-enhancer reporter activity (right) are increased by low-dose RA. Gates outlined in red denote EGFP⁺ cells (n=5-6). (Panel C) The TCF-LEF switch during the telogen-to-anagen transition is compartmentalized to the outer root sheath (ORS) and growing population of ‘transient amplifying cells’ (TACs) derived from the hair germ (HG). The epidermis-dermis interface is outlined; asterisks refer to autofluorescence in the sweat gland; arrows refer to LEF1⁺ ORS cells. Scale bar, 50 μm. (Panel D) The TCF-LEF switch is accurately modeled in atRA-treated HFSCs by exposure to Rspo, with associated SOX9 downregulation (left). Quantification of Sox9 mRNA following Rspo treatment (right; n=6). (Panel E) atRA is required for a Rspo-dependent increase in cultured HFSC proliferation, quantified by flow cytometry. Gates outlined in red denote EdU⁺ cells (n=6). (F) Wild-type mice were treated daily with topical RA or RARi during 2^{nd} telogen (top). RA treatment accelerated the onset of anagen entry, as measured by EdU incorporation in IF images (left) and quantified by flow cytometry (right; n=5). Gates outlined in red denote EdU⁺ cells. Scale bar, 50 μm.

FIG. 3: Directed differentiation to HF lineages via combinatorial niche signaling. (Panel A) Association of WNT-BMP co-activation with the emergence of CUX1⁺ transient amplifying cells (TACs) in the downgrowing HF (left). The epidermis-dermis interface is outlined; asterisk refers to autofluorescence in the sweat gland. Scale bar, 50 μm. (Panel B) CUX1 was upregulated by BMP in WNT-activated HFSCs (left) and WNT-mediated Lgr5 and Cux1 expression by qPCR was distinguished by low and high BMP activity, respectively (right; n=6). (Panel C) Emergence and maturation of major HF lineages, GATA3⁺ IRS and HOXC13⁺ HS, during HF downgrowth. The epidermis-dermis interface is outlined. Scale bar, 50 m. (Panel D) WNT-BMP co-activation upregulated lineage-specific Cux1 (IRS) and Cux1 (HS) reporter activity in cultured HFSCs, quantified by flow cytometry (left; n=5) and GATA3 and HOXC13 levels by immunofluorescence (right). HFSC colonies are outlined. Scale bar, 20 μm. TAC differentiation was promoted by lineage plasticity resolution and retinoic acid receptor inhibition (RARi). (Panel E) Wild-type mice were treated daily with topical RA or RARi during HF downgrowth (top). RA treatment prevented TAC differentiation, reducing the proportion of HOXC13⁺ cells relative to the GATA3⁺ IRS (left; n=10-12 HFs per sample) and prevented hair regrowth (right). The epidermis-dermis interface (left) and topical treatment area (right) are outlined. Scale bar, 50 μm.

FIG. 4: Transcriptional similarity of cultured HFSCs to the in vivo HF. (Panel A) Scheme depicting the diverse array of cell fates in the regenerating HF. (Panel B) UMAP clustering of cultured HFSCs treated with atRA and PKCi and exposed to Rspo, BMP, and RARi, placed in a shared embedding with a reference dataset of autologous, FACS-purified HF cells using Harmony (left; 5,333 cultured cells, 6,007 in vivo HF cells). Shifts in cultured HFSC identity, mapped onto annotated clusters, were consistent with the establishment of HFSC/ORS traits, Rspo-mediated HFSC activation, and differentiation following WNT-BMP co-stimulation, respectively (left). (Panel C) Reconstruction of cultured HFSC differentiation using Monocle 3 showed that BMPs drove positioning along pseudotime. Gene signatures derived using scRNA-seq data from Yang et al. (37) verified that cultured HFSCs advanced through a TAC-like state before terminal differentiation predominantly to the HS lineage. (Panel D) Differential gene expression (DEG) of cultured HFSCs across culture conditions identified ‘signature genes’ (right) and selected gene ontology (GO) terms (left; adj. p-value <10E-3) that accurately reflect the directed stage of HFSC differentiation.

FIG. 5: RA directly promotes HFSC fate during wound repair. (Panel A) Scheme depicting the steps taken and critical enzymes involved in RA synthesis, degradation, and transcriptional activity. (Panel B) Localized RA production in the HF via the ALDHA1 enzymes is disrupted during wound repair, revealed by IF (left) and flow cytometry using a fluorescent probe detecting ALDH enzymatic activity (right; n=4). The epidermis-dermis interface is outlined. Scale bar, 50 μm. (Panel C) Transduction of the surface epithelium in utero with lentiviruses delivering a construct designed to label and inducibly overexpress Cyp26b1 in Sox9-expressing HFSCs after Cre recombination (left). Detection of RFP⁺ Cyp26b1-overexpressing cells present in the uninjured epidermis and HF but overrepresented in the repaired epidermis post-wound by IF (center) and flow cytometry (right; n=11). The epidermis-dermis interface is outlined; arrows refer to YFP⁺RFP⁺ cells of HFSC origin and overexpressing Cyp26b1. Scale bar, 50 μm. (Panel D) YFP⁺ HFSCs inducibly labeled by the Sox9-CreER transgene in mice fed a vitamin A-deficient diet are traced throughout their contribution to epidermal barrier restoration or HF regeneration following a dermabrasion wound (top). Topical RA during the first week post-wound accelerated lineage plasticity resolution (left, n=6) and skewed HFSC contribution to hair regeneration, as measured by flow cytometry (right, n=6-10), while vitamin A loss had the opposite effect and slowed HF regrowth at intermediate stages of repair (center, n=9-12 HFs). Hair regeneration and HFSC fate were rescued by topical RA treatment.

FIG. 6. Panel A. Images showing serum replacement stabilized HFSC growth rate. Panel B. Graphics showing the influence of HFSC mitogens. Panel C. Graphs showing serum is not necessary or sufficient for lineage plasticity. Panel D. Graph showing colony formation changes. Panel E. Graphs showing results from targeting wound-related or HF niche signaling does not resolve lineage plasticity.

FIG. 7. Panel A. Graphs showing 3D cultures are required for HFSC proliferation. Panel B. Graphs showing serum prevents lineage plasticity resolution. Panel C. Graph and image shown RA and PKCi suppress the wound signature. Panel D. Graphs showing RA and PKCi target wound and epidermal hallmarks. Panel E. Graph and image showing RA uniquely upregulates HFSC markers. Panel F. Graphs showing RA supports key HFSC lineage TFs.

FIG. 8. Panel A. Chart and graphics showing identification of a ‘lineage plasticity’ gene signature from bulk transcriptomic comparison. Panel B. Graphical depictions showing reduced epidermal differentiation and cancer signatures following lineage plasticity resolution.

FIG. 9. Panel A. Image and graphs showing BMPs promote cell cycle exit in HFCs. Panel B. Graph showing BMP6 is most potent. Panel C. Graphs showing the effect of RA on BMP-mediated quiescence is dose-dependent.

FIG. 10. Panel A. Graphs showing BMP6 upregulates TcF3/4. Panel B. Graphs showing Rspo downregulates atRA-dependent Tcf3/4. Panel C. Graphs and images showing Rspo induces LEF1/TCF1 irrespective of RA. Panel D. Graph showing RA is dose-dependent. Panel E. Graphs showing RA and BMP inhibition cooperate with Rspo to upregulate Lgr5 expression.

FIG. 11. Panel A. Graphs showing effect of WNT-BMP co-stimulation in HFCS. Panel B. Graphs showing WNT is required for BMP-induced differentiation. Pane C. Image and graph showing BMP-induced HS and IRS differentiation. Panel D. Graphs showing BMP/24 dive Cux1-SE reporter activity. Panel E. Graphs showing spatial distribution of JS and IRS cells in HFSC colonies.

FIG. 12. Images showing de novo hair folliculogenesis following cultured HFSC transplantation.

FIG. 13. Panel A. Graphical depictions of measurements based on clustering of autologous cultured and in vivo datasets following integration. Panel B. Graphic showing prediction scores for cultured HFSCs relative to in vivo clusters. Panel C. Charts showing gene ontology enrichment of DEGs between cultured and in vivo HFSCs.

FIG. 14. Panel A. Graph showing atRA is most bioactive metabolite. Panel B. Graphs showing Cym26b1 overexpression comprises HFSC identity and behavior. Panel C. Graphic and graphs showing cell fate of wild-type and Cyp26b-1OE HFSCs. Panel D. Graphs showing lentiviral delivery is not biased to HFSCs or EpSCs prior to induction.

FIG. 15. Panel A. FACS plots showing Sox9^{CreER} skin flow gating strategy. Panel B. Image and graph showing vitamin A loss impacts HFSC fate during wound repair. Panel C. Images showing vitamin A loss impacts HFSC fate during wound repair. Panel D. Graph showing trans-epidermal water loss. Panel E. Graphs showing skin retinol depletion following a vitamin A-deficient diet.

FIG. 16. Panel A. Graphic and image showing topical RA manipulation during wound repair. Panel B. Graphs showing RA activity promotes HFSC retention and hair regeneration.

FIG. 17. Images and graphs obtained using standard HFSC media and conditions for culturing HFSCs in 3D as described in the detailed description.

FIG. 18. Graphs showing were initially established base conditions for HFSC growth and survival in 3D cultures and modifications screened for their ability to reduce lineage plasticity as measured by a Klf5-EGFP transgene as described in the detailed description.

FIG. 19. Images and graphs showing HFSC growth patterns and overall colony structure were also affected by the presence of serum as described in the detailed description.

FIG. 20. Images and graphs showing titration of atRA (10 nM-1 μM) and PKCi (0.1-10 μM) to determine the optimal concentration to reduce lineage plasticity and promote physiological HFSC behavior as described in the text.

FIG. 21. Images showing analysis of the translatability of the described mouse methodology enabling the faithful recapitulation of HFSC identity in culture to humans by introducing FBS, B-27 and atRA+PKCi conditions into human keratinocytes as described in the detailed description.

### DETAILED DESCRIPTION

As used in this disclosure including the claims, the singular forms “a”, “an”, and “the” include plural references unless the context clearly dictates otherwise. Thus, for example, references to “the method” includes one or more methods, and/or steps of the type described herein, which will be apparent to those persons skilled in the art having the benefit of this disclosure.

Unless defined otherwise herein, all technical and scientific terms used in this disclosure have the same meaning as commonly understood by one of ordinary skill in the art to which this disclosure pertains.

Where a range of values is provided in this disclosure, it should be understood that each intervening value, to the tenth of the value of the lower limit of that range, and any other intervening value and ranges in that stated range are encompassed within the disclosure, unless clearly indicated otherwise. The upper and lower limits of these smaller ranges may independently be included in the smaller ranges encompassed within the disclosure.

The disclosure includes all steps and compositions of matter described herein in the text and Figures of this disclosure, including all such steps individually and in all combinations thereof, and includes all compositions of matter and methods of using the same as described herein. Any step and/or reagent described herein may be performed and/or used sequentially or concurrently. Any cell culture media described herein may comprise or consist of the described components. Any individual media component or combination thereof may be included, or excluded from the claims of this disclosure. In examples, a cell culture media and a method of using the media may be viral vector free, such as free of any viral vector that is used to induce any stem cell phenotype, and/or be serum free.

In examples, the disclosure provides methods for producing cells that express the described protein and/or exhibit a gene expression profile, as further described herein. It will be recognized that the described protein and expression of their genes may be specific to a particular state of cellular development.

In examples, the present disclosure provides a cell, and populations of cells produced by the methods described herein. The cells can be used fresh after culture or can be frozen for later use. Cell lines produced by a described method are also provided. The cells used in the described methods may be any type of mammalian cells. In examples the cells are human cells.

In one example, the disclosure provides an in vitro method of generating hair follicle stem cells, the method comprising: culturing epithelial cells in a cell culture medium comprising all-trans-retinoic acid (atRA) and a calcium-dependent protein kinase C inhibitor (PKCi) to thereby promote the epithelial cells to become the hair follicle stem cells. In an example, the PKCi specifically inhibits PKC calcium-dependent alpha and beta isoenzymes. In an example, the PKCi is Go-6976. In an example, the cell culture medium further comprises one or more of Dulbecco's Modified Eagle Media/Ham's F12 media (DMEM/F12), Calcium Chloride, Y-27632, Sodium bicarbonate, cholesterol-cyclodextrin, L-glutamine, or a mitogen. Suitable mitogens include but are not necessarily limited to fibroblast growth factor 10 (FGF10) or epidermal growth factor (EGF). In an example, the mitogen is FGF10. In an example the cell culture medium further comprises B-27 supplement without vitamin A; or a mixture comprising one or a combination of:
Palmitate ((bovine serum albumin) BSA-conjugated))
Oleate (BSA-conjugated)
Linoleate (BSA-conjugated)
DL-a-tocopherol acetate
L-carnitine
Sodium selenite
Glutathione
T3
Apo-transferrin, and
Insulin.

B-27 is a known cell culture component and is commercially available, such as THERMOFISHER Scientific. In an example, B-27 is as described in Brewer et al., J Neuroscience Res 35: 567-576, 1993 and Brewer and Cotman, Brain Res 494: 65-74, 1989, from which the disclosures are incorporated herein by reference. The disclosure includes an isolated population of hair follicle stem cells made by the described methods, using the described cell culture medium.

Go-6976 is a commercially available compound with a known structure (CAS No. 136194-77-9). Y-27632 is a commercially available compound with a known structure (CAS No.:146986-50-7).

Any method of producing hair follicle stem cells using a described culture medium can be compared to another method, which may be considered a reference method. A method of the disclosure that uses a described culture medium may produce a different or improved result compared to a method that uses reference method, including but not necessarily limited to any of: faster development of hair follicle stem cells than the reference method; production of more hair follicle stem cells than the reference method; or improved control over differentiation of the hair follicle stem cells compared to the reference method.

In one example a method of the disclosure comprises culturing isolated hair follicle stem cells made according to this disclosure in serum-free media primed with only one, or more than bone morphogenetic protein (BMP) protein, BMP6 R-spondin, BMP4, BMP6, or AGN193109 (a representative retinoic acid receptor inhibitor). Using one or a combination of these described agents may promote formation of different cell states, such as hair follicles, or quiescence cells, or another cell fate that is not necessarily a hair follicle. Specifically, BMP6 when presented with a relatively low concentration of atRA (10 nM) resulted in cell cycle exit and maintained a state resembling quiescence. R-spondins in combination with a 10-fold greater concentration of atRA instead drove lineage progression to a more proliferative, transient amplifying cell fate. R-spondins, BMP2, and BMP4 directed HFSCs to form the hair shaft and inner root sheath lineages of the differentiated hair follicle. Hair shaft differentiation was uniquely promoted by the addition of AGN193109. AGN193109 is commercially available and its structure is known.

In an example, the disclosure comprises comprising mixing isolated hair follicle stem cells produced according to a method of this disclosure with dermal fibroblasts. The method may further comprise engrafting the isolated hair follicle stem cells and dermal fibroblasts onto skin of an individual to thereby cells that are differentiated from the hair follicle stem cells.

In another aspect the disclosure comprises a method comprising exposing a test compound to an isolated population of hair follicle stem cells made according to the described methods and using a described cell culture media, and determining whether or not the test compound influences a property of the hair follicle stem cells. For example, a test compound may be tested to determine whether or not the hair follicle stem cells survive, or whether the hair follicle stem cells differentiate into a particular lineage. The test compound may be compared to, for example, a control compound that has a known effect on hair follicle stem cell differentiation. The test compound is not particularly limited, and may be any compound with a known or unknown function. In examples, combinations of test compounds may be used. In examples, the effect of a test compound is compared to a reference compound, the function of which is known or established before analyzing effects of the test compound.

In an example, the disclosure provides a culture medium for use with hair follicle stem cells. The cell culture media comprises atRA and a PKCi. The cell culture medium may further comprise one or more of DMEM/F12, Calcium Chloride, Y-27632, Sodium bicarbonate, cholesterol-cyclodextrin; L-glutamine; and a mitogen that is optionally FGF10 or EGF. The cell culture media may further comprise B-27 supplement without vitamin A; or a mixture comprising:
Palmitate (BSA-conjugated)
Oleate (BSA-conjugated)
Linoleate (BSA-conjugated)
DL-a-tocopherol acetate
L-carnitine
Sodium selenite
Glutathione
T3
Apo-transferrin, and
Insulin.

With respect to developing the describe cell culture media, to overcome previous issues and successfully propagate bonafide HFSCs outside the body absent their native niche, the disclosure relates in part to the observation herein that widespread chromatin remodeling in HFSC cultures is analogous to the early stages of wound repair (R. C. Adam, H. Yang, S. Rockowitz, S. B. Larsen, M. Nikolova, D. S. Oristian, L. Polak, M. Kadaja, A. Asare, D. Zheng, E. Fuchs, Pioneer factors govern super-enhancer dynamics in stem cell plasticity and lineage choice. Nature. 521 (7552): 366-70 (2015)). Injury imposes a reversible state of lineage plasticity to HFSCs, enabling the transition from one committed developmental skin lineage to another to more rapidly repair the damaged tissue. This is controlled by the transcription factors of both the new and prior lineages as well as wound-induced stress factors. We analyzed whether identifying and targeting the signals that drive the wound response in culture could resolve lineage plasticity, and determined an a defined set of culture conditions that permit the re-acquisition and long-term maintenance of HFSC identity. Representative examples of the culture conditions are described further below. As discussed above, the disclosure includes improvements that include but are not necessarily limited to media modifications and that include the use of all-trans retinoic acid (atRA) and an inhibitor of protein kinase C (PKCi) in defined concentrations.

With respect to the presently provided improved methods, other work generated mouse and human hairy skin organoids that also maintain HFSCs. (J. Lee, et al., Generation and characterization of hair-bearing skin organoids from human pluripotent stem cells. Nat Protoc, 1-40 (2022); and K. R. Koehler, et al., Generation of inner ear sensory epithelia from pluripotent stem cells in 3D culture. Nature. 500, 217 (2013). These methods aggregate pluripotent stem cells into embryoid bodies and direct their specification to surface ectoderm, driving the formation of epithelial cysts that spontaneously grow complex skin harboring HFSCs. These methods are distinct from those of the present disclosure, which instead show the generation of HFSCs in the context of fully formed hair and a suite of niche cell types normally present in skin. Prior methods also risk residual pluripotent stem cell contamination if used for direct therapeutic application, a significant safety issue, and are more laborious, requiring months to produce HFSCs. In contrast, the presently described conditions restore HFSCs to their physiological state in a matter of days and can facilitate patient-specific preclinical testing of existing and new therapeutic modalities designed to treat hair and skin disorders in settings where time is of the essence.

In examples, a presently described cell culture or population of cells is free of pluripotent stem cells. The disclosure includes the proviso that practicing a method of the disclosure may be performed without aggregation of pluripotent stem cells into embryoid bodies. The disclosure includes the proviso that practicing a method of this disclosure may be performed such that no formation of epithelial cysts occurs.

To investigate how lineage plasticity is resolved with respect to hair follicle stem cells, we analyzed the mammalian skin epithelium, a barrier tissue that has evolved to rapidly heal in response to environmental insults. The skin epithelium contains several discrete stem cell (SC) populations derived from a common embryonic progenitor. During homeostasis, interactions with their niches specialize the tasks that each SC performs. SCs within the ‘bulge’ niche of each hair follicle (HF) constitute the engine for cyclical bouts of HF regeneration and hair growth, while epidermal stem cells (EpSCs) within the innermost (basal) layer of the interfollicular epidermis generate the flux of upwards stratifying cells that rejuvenate the skin's barrier to the outside world.

Upon injury, HFSCs and EpSCs are mobilized from their niches to migrate into the wound bed where they enter a state of lineage plasticity, essential to re-epithelialize the denuded epidermis. This is supported by the co-activation of Sox9 and Klf5, encoding the master regulatory transcription factors that distinguish HFSCs and EpSCs, respectively. HFSCs are notable in that following injury, their normal regenerative task of regrowing the hair follicles (HFs) becomes repurposed to re-epithelialize the wounded epidermis.

HFSCs enter a similar wound-like state when placed in culture, where they co-express SOX9 and KLF5 and undergo widespread chromatin remodeling characteristic of lineage plasticity. By exploiting this phenomenon, the present disclosure reveals retinoic acid (RA) at the root of stem cell lineage plasticity. Surprisingly, RA-mediated resolution and repression of epidermal fate was required to permit HFSCs to undergo hair lineage progression in culture. In vivo genetics confirmed RA levels as a powerful controller of stem cell contributions to both homeostasis and wound repair. Thus, and without intending to be bound by any particular theory, it is considered the present disclosure reveals the missing link between retinoids and lineage plasticity, both important to skin biology and tissue regeneration but whose specific functions until the present disclosure have remained elusive.

The following Examples are intended to illustrate various examples of the disclosure but are not meant to be limiting.

### Example 1

### Resolution of Lineage Plasticity in HFSCs Ex Vivo

Normally exclusive to EpSCs, Klf5 is transiently induced by HFSCs in response to injury, when they migrate into the wound site and participate in epidermal repair. When activated in HFSCs, KLF5 becomes a hallmark of lineage plasticity. To restore homeostasis and return to devoting their energy to hair regeneration, HFSCs suppress KLF5 and the genes that it regulates.

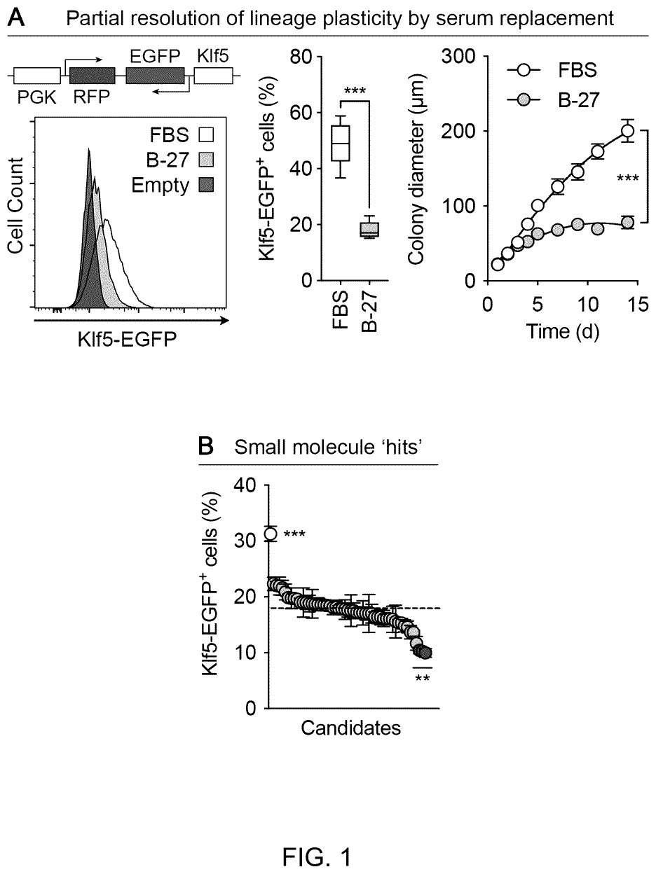

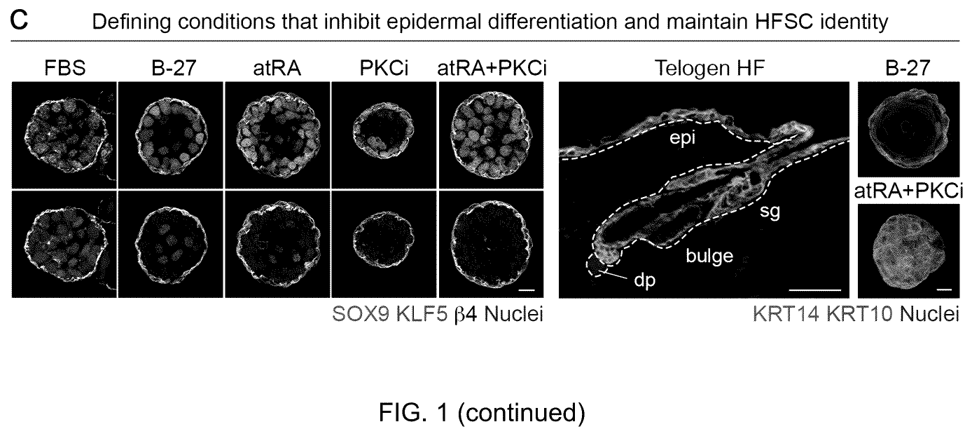

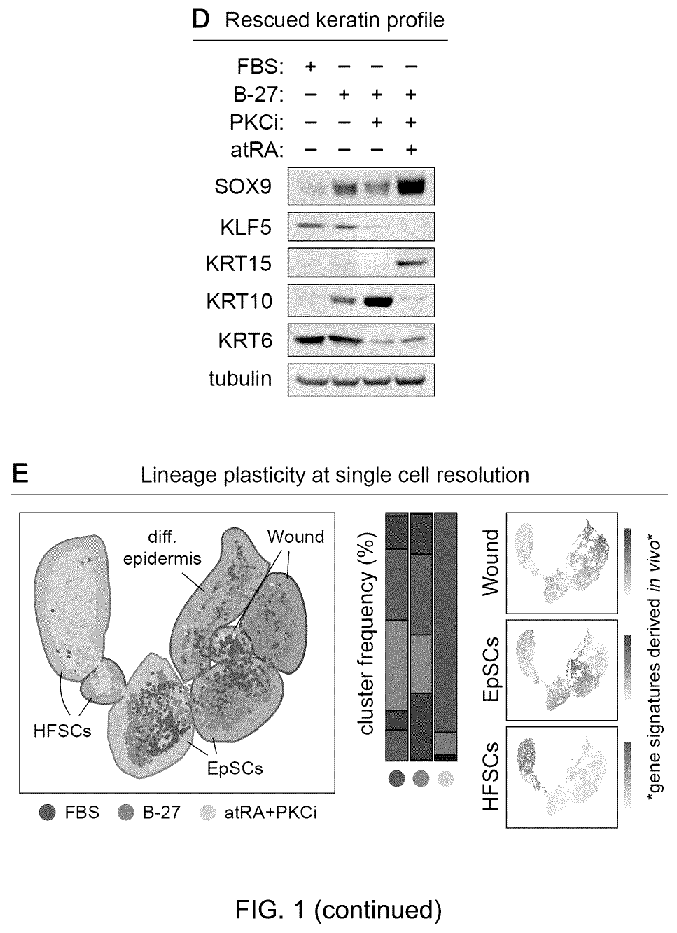

To identify the previously elusive signals that maintain HFSC identity and suppress lineage plasticity as HFSCs return to homeostasis, we first generated a lineage plasticity reporter system. Purified HFSCs were stably transduced by lentivirus harboring a Klf5-EGFP-NLS transgene and transduction control (Pgk-RFP-NLS) and grown in a modified 3D culture system (FIG. 1 Panel A).

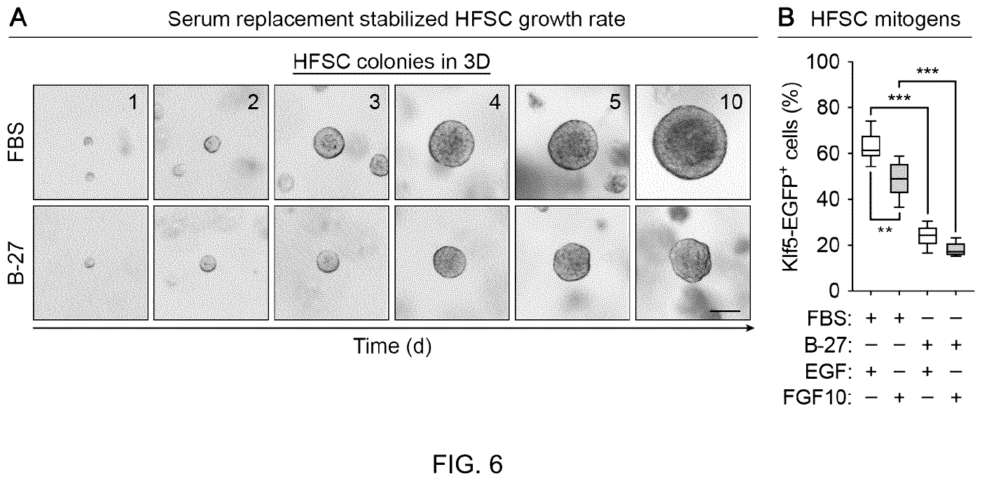

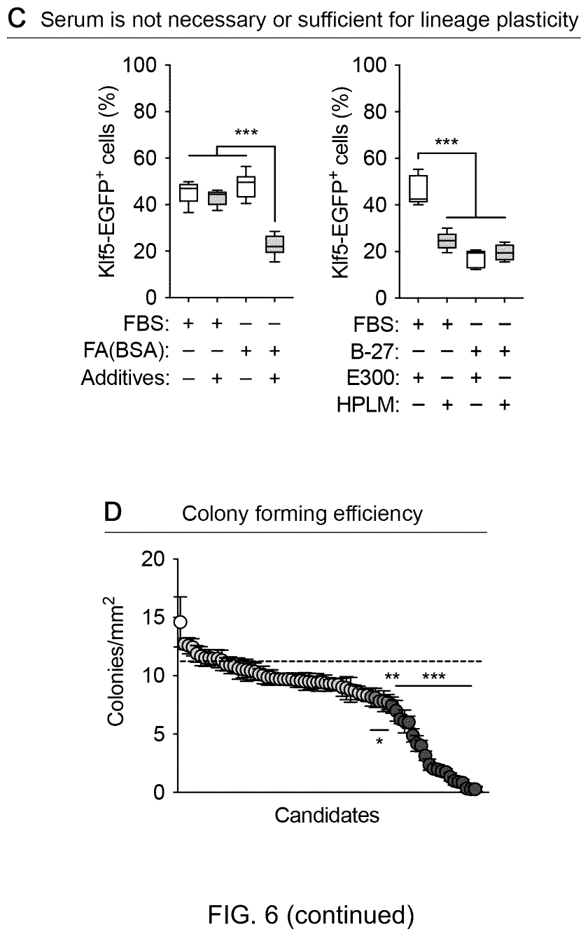

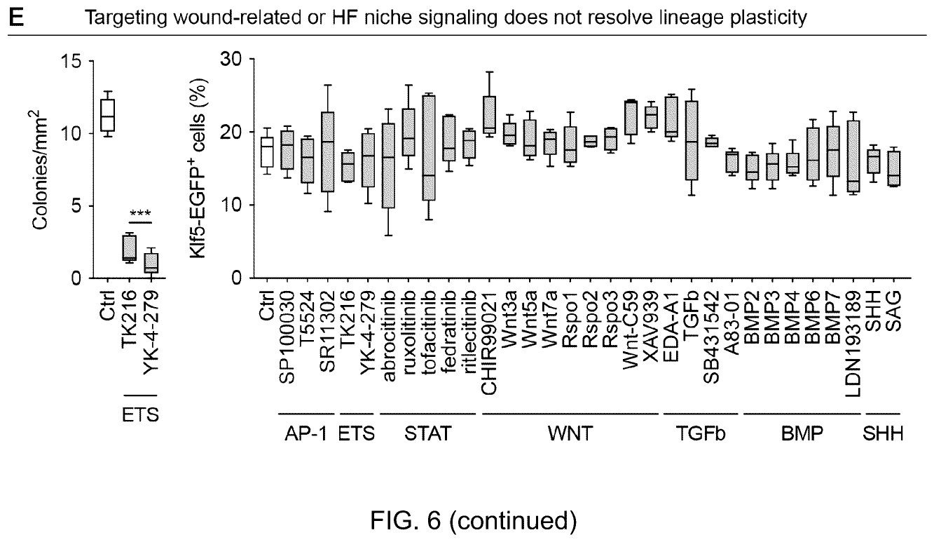

By replacing fetal bovine serum (FBS) with a serum-free media supplement (B-27) and using the native HFSC niche mitogen FGF10, Klf5-EGFP levels were partially reduced and unchecked colony growth was stabilized without compromising colony forming efficiency (FIG. 6, Panels A and B). Serum itself was not needed to support lineage plasticity, as replacement with a cocktail of fatty acids and cholesterol did not reduce Klf5-EGFP levels without antioxidants (FIG. S1 Panel C). Media formulated to mimic human plasma resulted in reduced Klf5-EGFP levels even in the presence of serum. These results underscore the complexity of signaling networks involved in suppressing lineage plasticity.

To further reduce Klf5-EGFP levels in serum-free conditions, we targeted pathways associated with lineage plasticity, initially focusing on the AP-1, ETS, and STAT families. However, selective inhibitors to these pathways did not reduce Klf5-EGFP levels without negatively affecting colony formation (FIG. 6, Panels D and E). Similarly, we tested agonists and antagonists of core signaling cascades, including members of the WNT, TGFβ, BMP and SHH pathways involved in lineage progression of HFSCs during hair follicle and hair regeneration. All failed to significantly affect Klf5-EGFP levels or resolve lineage plasticity (FIG. 6, Panels D and E).

To define historically elusive mediators of lineage plasticity, we screened a library of small molecules that have been broadly implicated in wounding but had no prior link to regulating the lineage plasticity step required for re-epithelialization. Of >70 compounds tested (Table A), a competitive inhibitor of extracellular signal-regulated kinases (ERKs) and mitogen activated protein kinases (MAPKs), calcium-dependent protein kinase C inhibitor (PKC), and the major active metabolite of retinol/vitamin A₁ in skin, all-trans-retinoic acid (atRA), proved particularly effective agents impacting lineage plasticity in cultured HFSCs (FIG. 1 Panel B). These factors had been previously implicated in either wound repair and/or hair growth, but their precise roles in these processes were ambiguous. Given the general role of the ERK/MAPK pathway in activating the RAS and promoting proliferation, we instead focused our attention on the less well understood and more specific effects of PKCi and atRA.

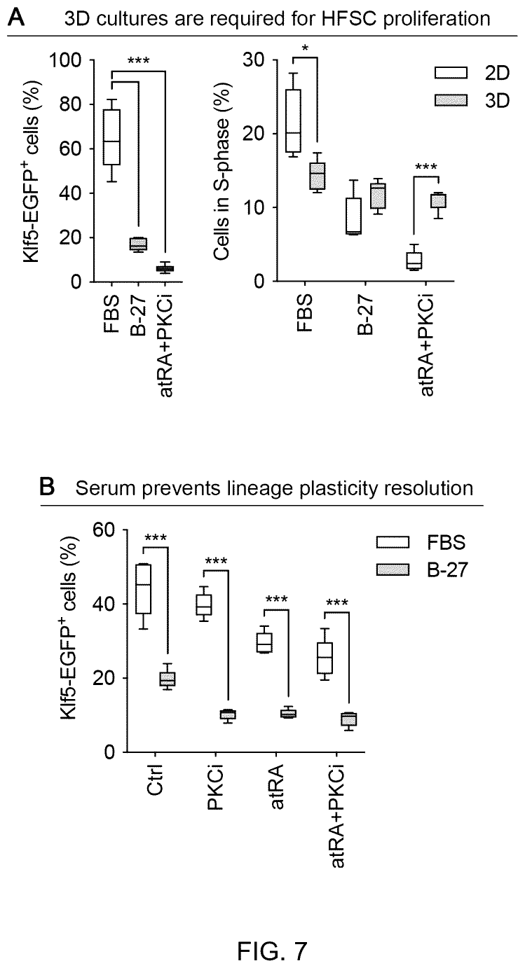

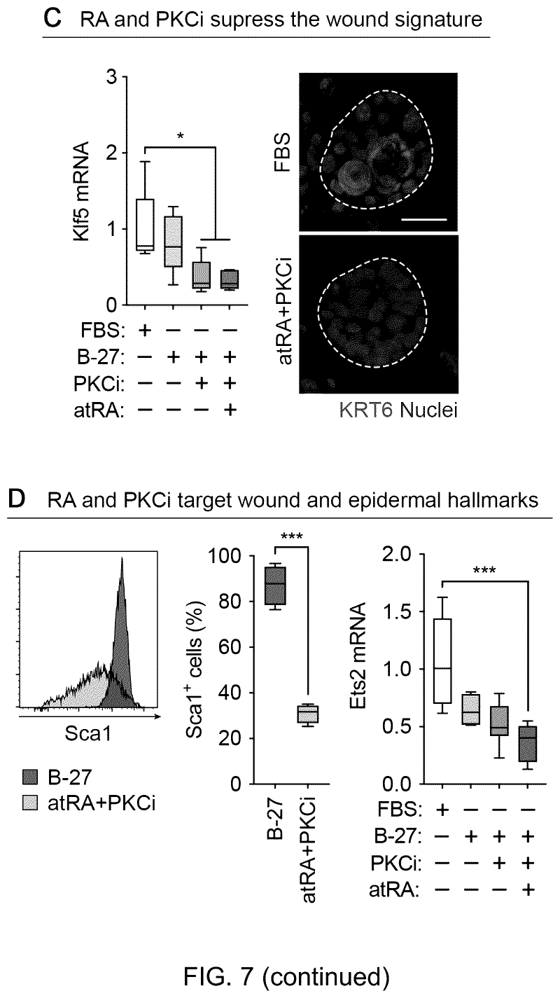

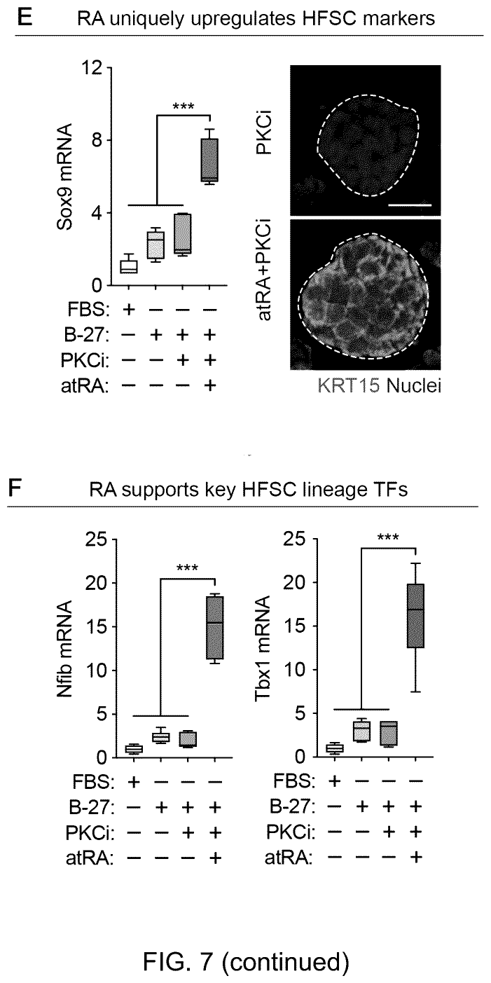

We first explored these two hits against the backdrop of our more physiological (serum-free) conditions, where SOX9 and KLF5 had already partitioned their expression peripherally and internally within colonies, respectively (FIG. 1 Panel C). PKC inhibition suppressed KLF5 expression. By contrast, although atRA was not as effective in reducing KLF5, it markedly expanded the SOX9 population. The combination of atRA and PKCi optimally resolved lineage plasticity and favored HFSC behavior, which was particularly evident in 3D culture conditions that mimicked the in vivo microenvironment of the HFSC niche (FIG. 7, Panels A and B).

Immunoblot, qPCR and additional immunofluorescence analyses strengthened the evidence that PKCi and atRA resolve lineage plasticity (FIG. 1 Panel D, FIG. S2, Panels C to E). Keratin KRT6 and Ets2, hallmarks of hyperproliferative and cancerous epidermis, were also suppressed by PKCi. Intriguingly, prominent HFSC identity markers, including SOX9 and KRT15, were dependent upon atRA, which correspondingly suppressed epidermal markers including Sca1 and KRT10. atRA also restored Nfib and Tbx1 expression, which help to maintain the HFSC epigenetic landscape and support self-renewal (FIG. 7 Panel F).

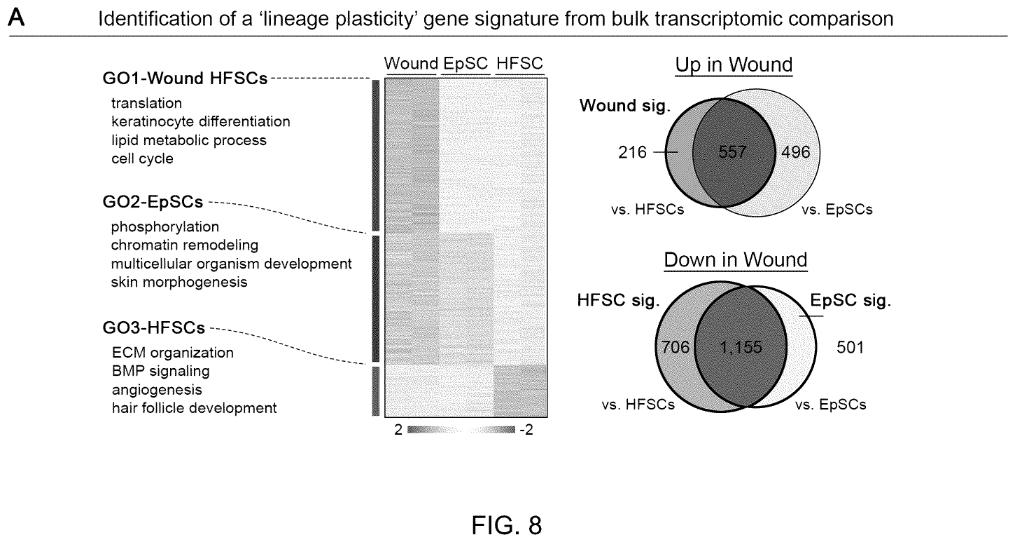

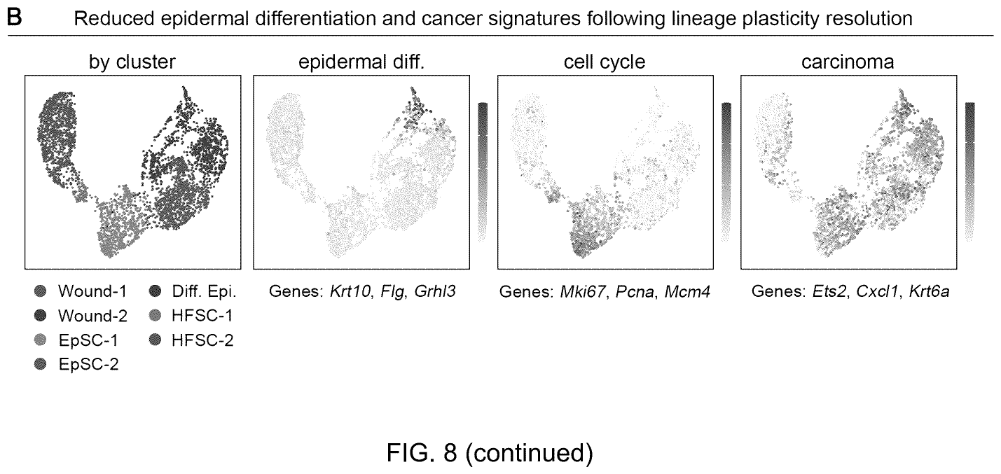

The unprecedented ability to achieve and maintain HFSC identity in vitro was exemplified more globally through single cell RNA-sequencing (scRNA-seq) of HFSCs exposed to these conditions (FIG. 1 Panel E). Leveraging existing bulk RNA sequencing data from EpSCs and lineage-traced HFSCs that had been isolated before and during wound repair in vivo, we obtained unique gene signatures for each stem cell type and condition and compared these to the signatures of the described cultured HFSCs (FIG. 8 Panel A). HFSCs cultured under the described new conditions formed a homogeneous cluster with a strong in vivo HFSC signature and nearly absent of wound or EpSC signatures (FIG. 1 Panel E). Notably, signature genes associated with strong enhancers active in skin squamous cell carcinomas (SCCs) and in the described prior serum-stimulated culture conditions were also dramatically reduced in the HFSC cluster (FIG. 8 Panel B). In total, the ability to suppress lineage plasticity with the described new culture conditions and the parallel suppression of wound- and cancer-associated signatures further underscore the overlapping roles of lineage plasticity in wounds and cancer.

### Example 2

### RA and BMPs Cooperate to Promote HFSC Identity

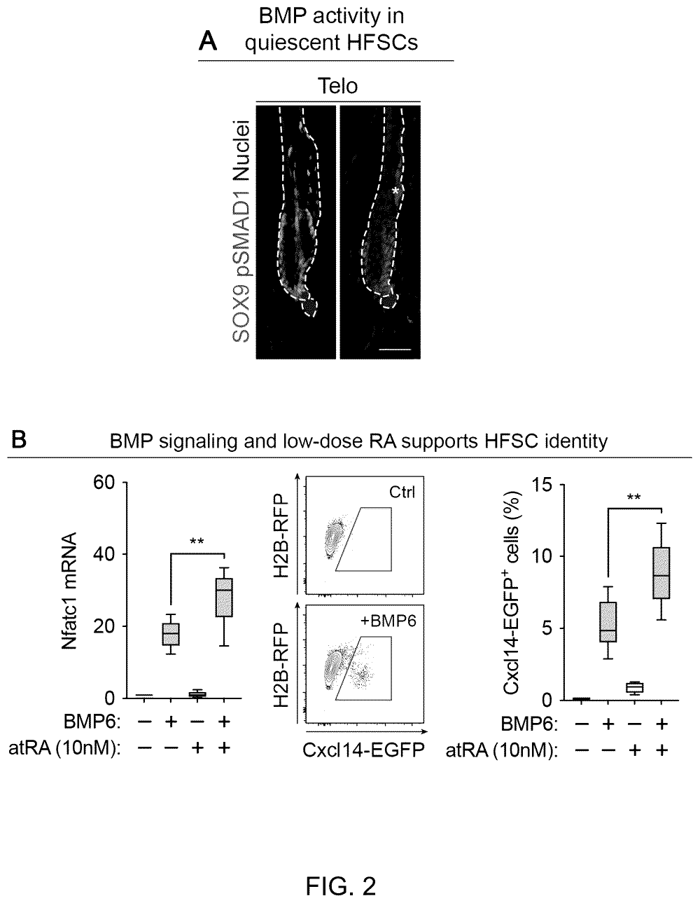

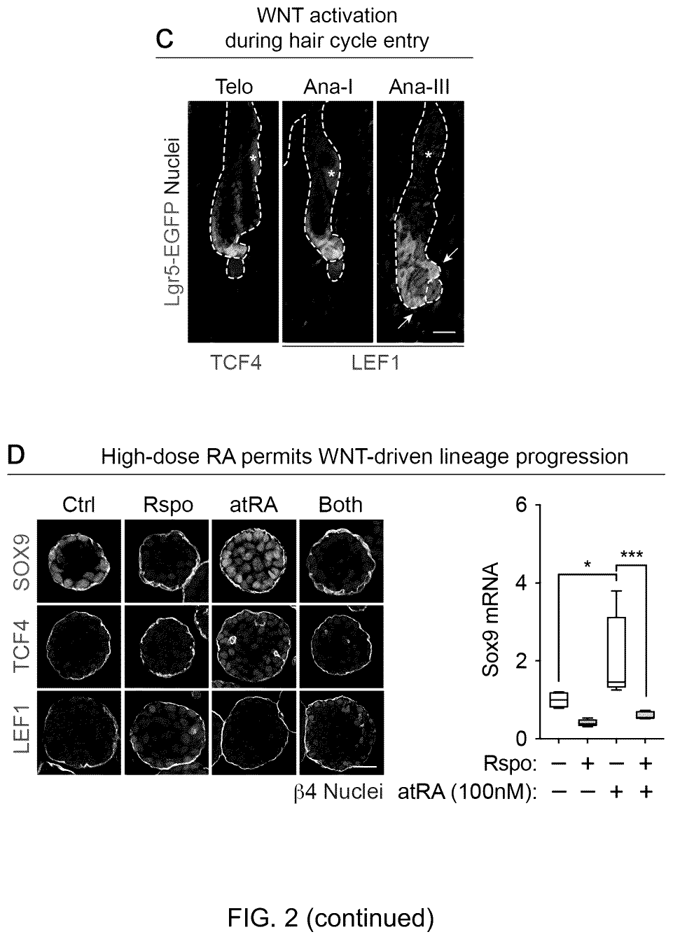

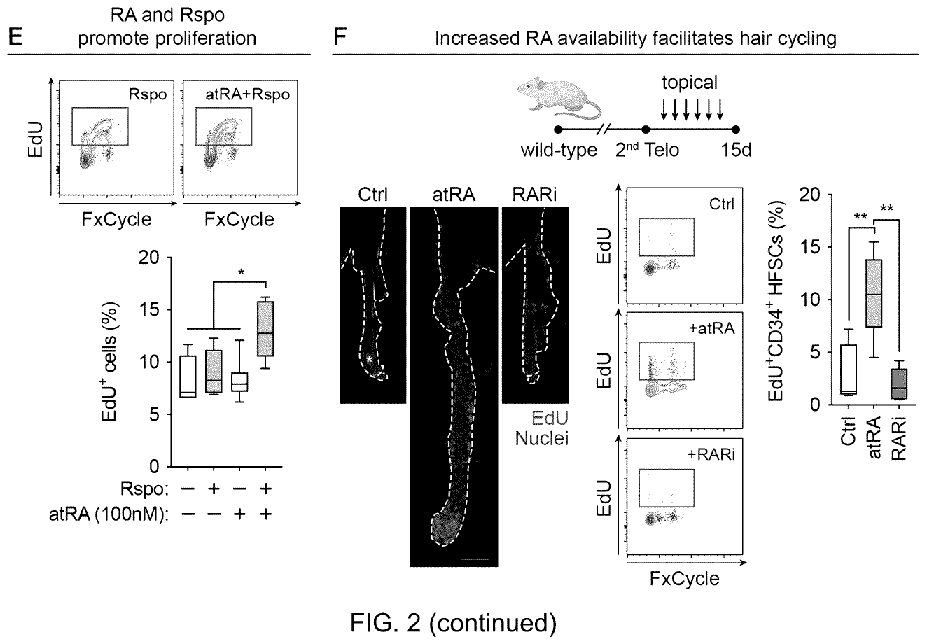

In vivo, HFSCs can be divided into two populations: those in a more quiescent state found in the bulge and ones primed for activation, located in the ‘hair germ’ (HG) just below the bulge. As BMP6 is produced by the inner bulge and responsible for maintaining HFSCs in their quiescent state (FIG. 2 Panel A), we continued to utilize a described culture system by assessing how these factors impact HFSC activation and fate commitment.

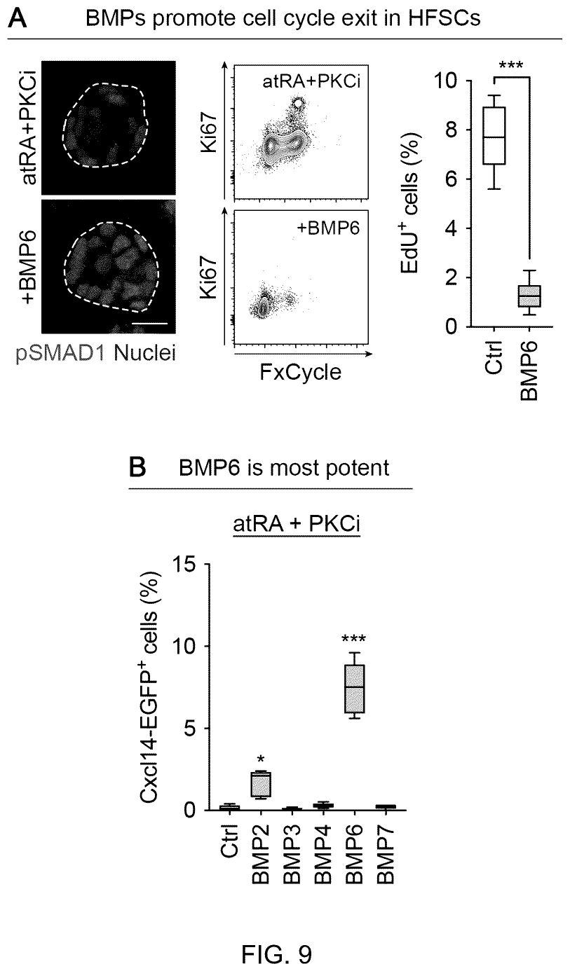

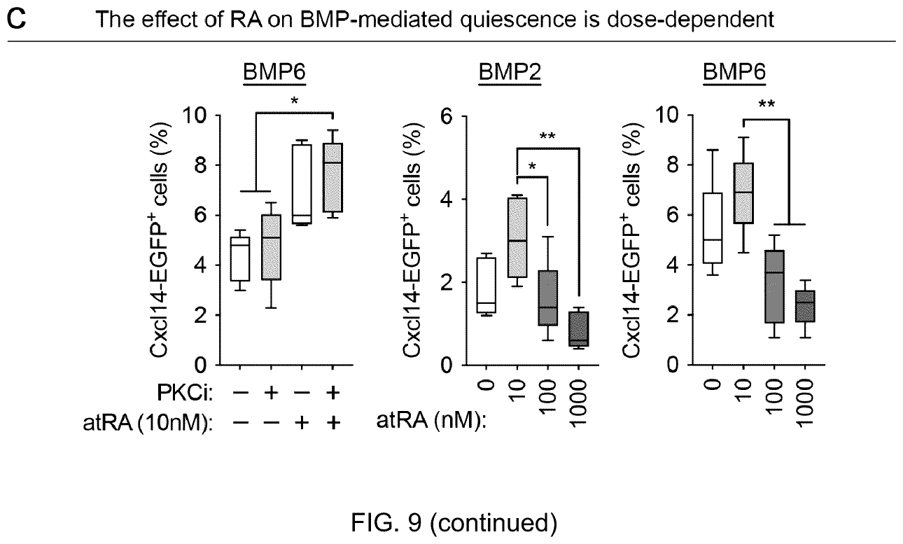

BMPs triggered cell cycle exit (FIG. 9 Panel A). BMP6 showed the most potent effect, as judged by expression of bulge gene Nfatc1, not expressed appreciably in prior culture conditions, and a fluorescent reporter driven by the Cxcl14 promoter, active in the bulge (FIG. 2 Panel B, FIG. 9 Panel B). Consistent with its role in reducing lineage plasticity but not affecting HFSC identity, PKCi exerted little or no effect on Cxcl14 activity. By contrast, atRA generated a dose-dependent boost in bulge HFSC identity, optimal at low concentration (FIG. 9 Panel C).

### Example 3

RA Functions with Wnts to Drive Hair Lineage Progression

The initiation of the hair cycle is controlled by inhibitors of BMP and activators of canonical WNT signaling emanating from the mesenchymal cells of the HG niche, the dermal papilla (DP). Activated HFSCs produce two progenitor populations: SOX9⁺TCF3/4⁺ cells in the outer root sheath (ORS) of the HF, the upper portion of which harbors the HFSCs for the next hair cycle, and short-lived, highly proliferative LEF1⁺TCF1⁺ transit amplifying cells' (TACs) that maintain contact with the DP in a WNT-rich and BMP-inhibited microenvironment and give rise to the hair shaft (HS) and its channel, the inner root sheath (IRS) (FIG. 2 Panel C).

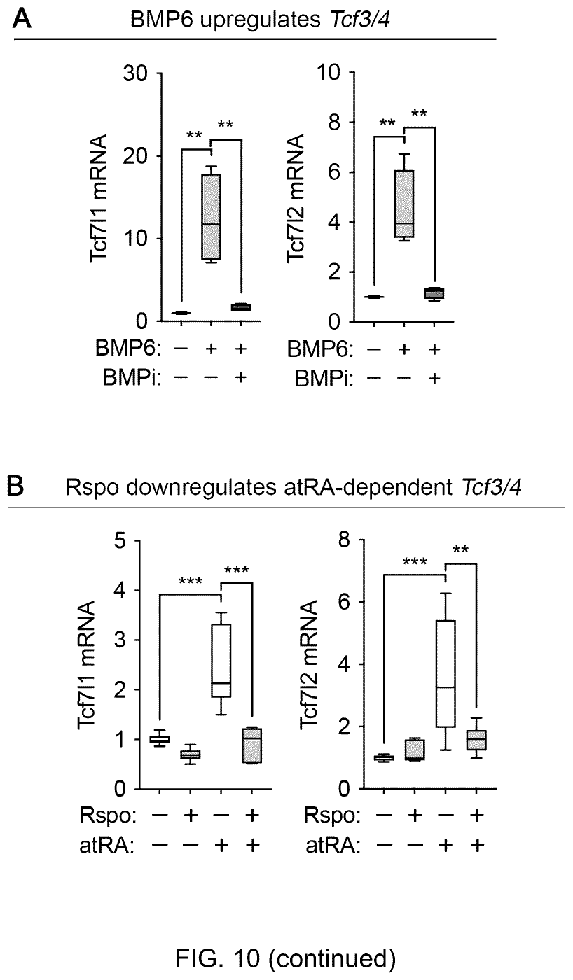

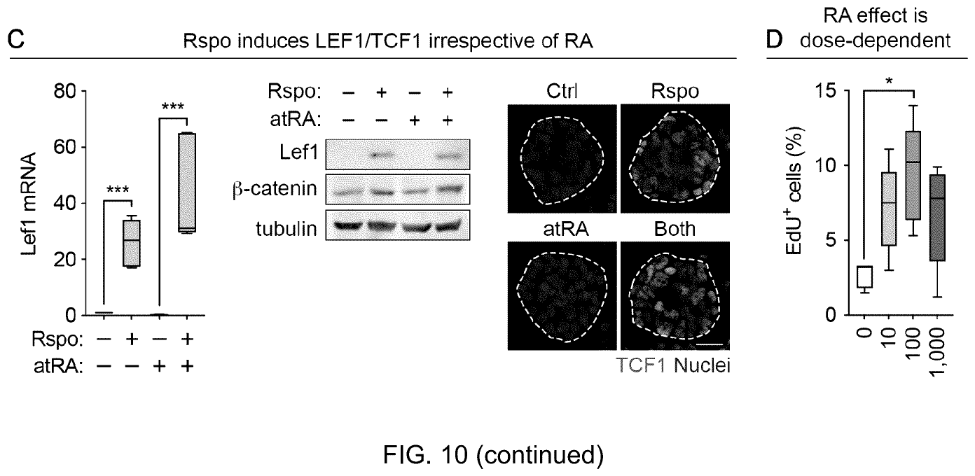

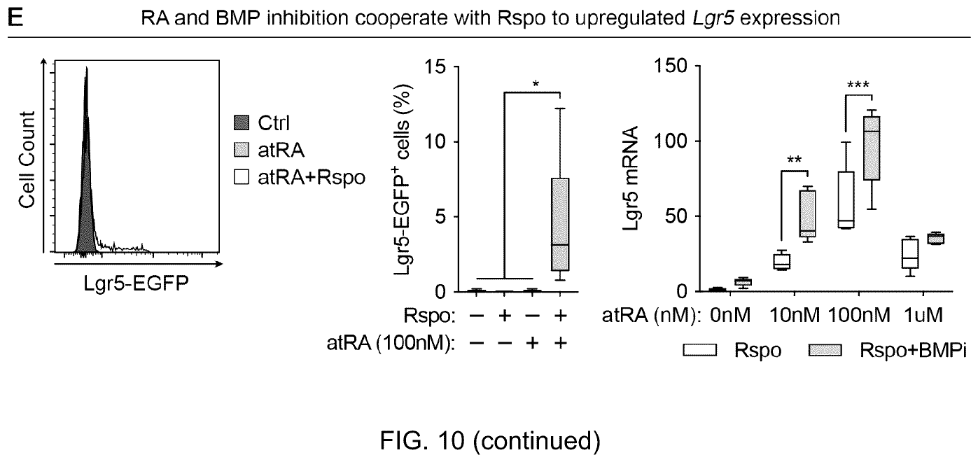

In vitro, none of the four WNT-regulating TCF/LEF transcription factors were expressed under standard serum conditions or in serum-free conditions, even with PKCi. Consistent with its newfound role in favoring HFSC identity, atRA promoted expression of Tcf7l1 (TCF3) and Tcf7l2 (TCF4) (FIG. 2 Panel D). These HFSC-associated TFs were also stimulated by BMP6, consistent with a raised threshold for activating WNT during quiescence (FIG. 10, Panels A and B). By contrast, R-spondins (Rspo), the native stimulators of canonical WNT signaling mediated the switch in vitro between stemness and TACs, downregulating SOX9/TCF3/TCF4 and inducing LEF1/TCF1 (FIG. 2 Panel D, FIG. 10 Panel C). Rspo-mediated HFSC proliferation was sensitive to atRA with the greatest cooperative effect seen at 10-fold higher the atRA concentration that had been optimal for quiescence-related features of stemness (FIG. 2 Panel E, FIG. 10 Panel D).

The in vitro data described above suggested that while atRA is essential for HFSC identity and stemness, its levels determine whether it will function with BMPs to maintain quiescence (low levels) or with Rspo to mediate HF regeneration (high levels). To test this in vivo, we topically applied atRA to the back skin of mice during the resting phase of the hair cycle and then followed the change in skin color, indicative of the concomitant melanogenesis that occurs upon hair cycle entry. atRA facilitated hair cycle entry, increasing EdU incorporation in CD34⁺ HFSCs and accelerating HF downgrowth at early time points after administration (FIG. 2 Panel F).

### Example 4

### Directed Differentiation to HF Lineages Via Combinatorial Niche Signaling

The lineages of the HF are as complex as those of the hematopoietic system, but in contrast, these lineages are spatially and temporally defined and hence intimately associated. With the described new insights into the HFSC niche microenvironment and our ability to suppress EpSC fate in culture, we began to dissecting the signaling pathways that have long confounded understanding of stem cell lineage progression in this mini-organ. Without intending to be bound by any particular theory, it is considered that for the first time, the disclosure reveals signals that directly impact HFSCs, independent of the myriad of secondary circuits and intercellular interactions that can operate within the native microenvironment of the skin and participate in hair regeneration.

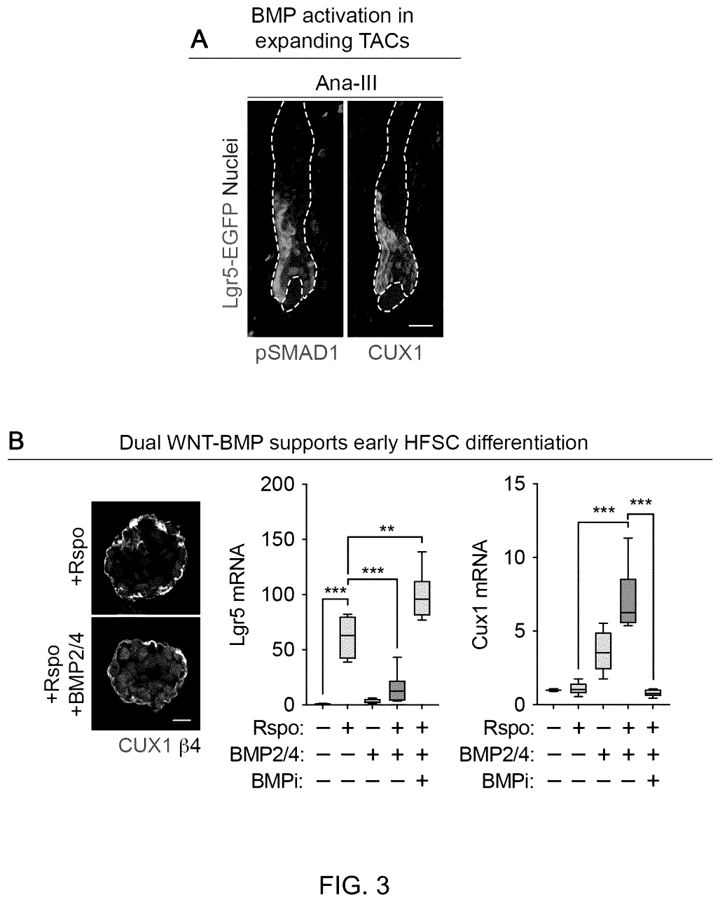

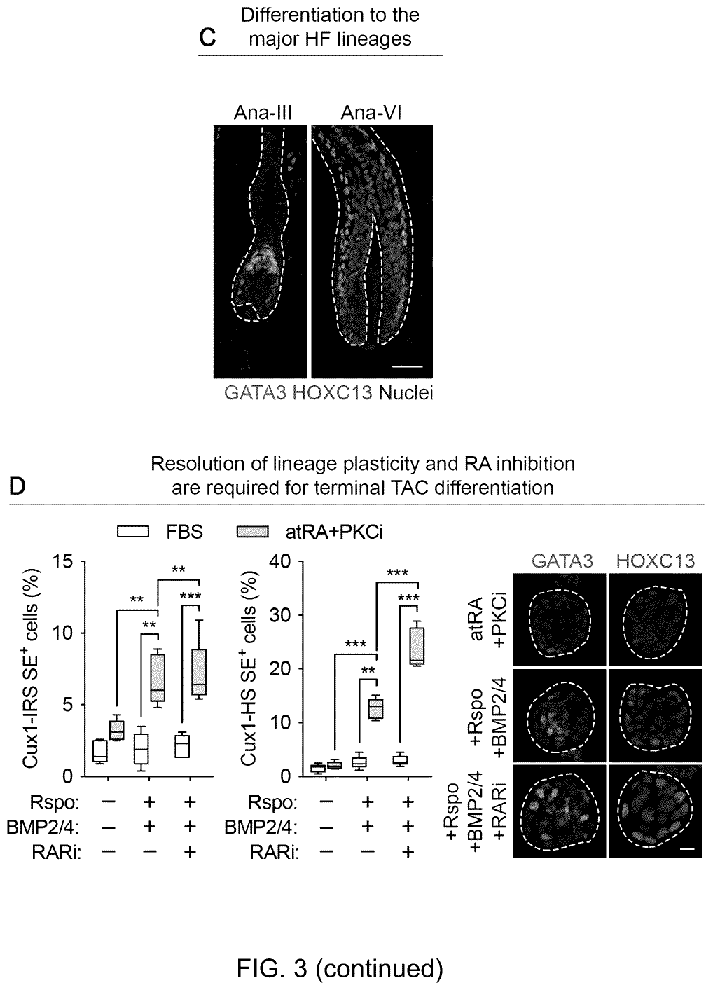

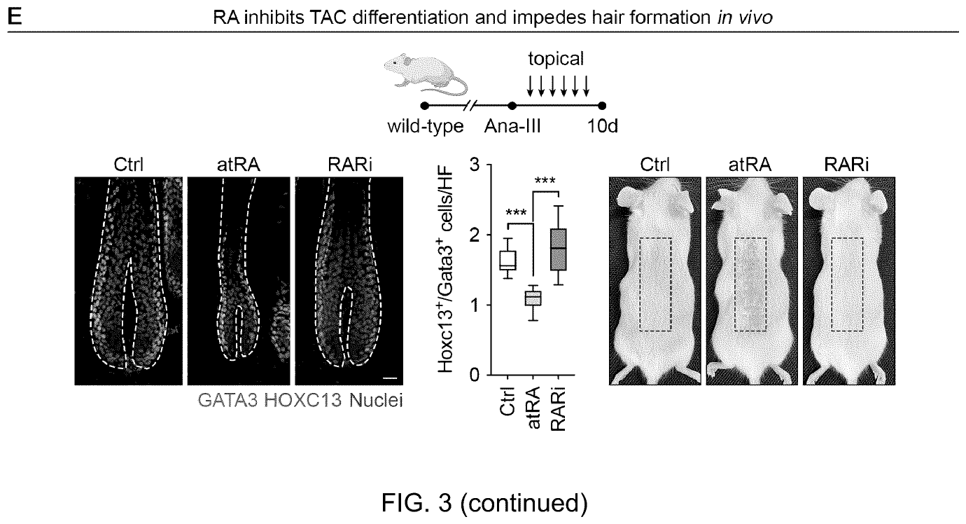

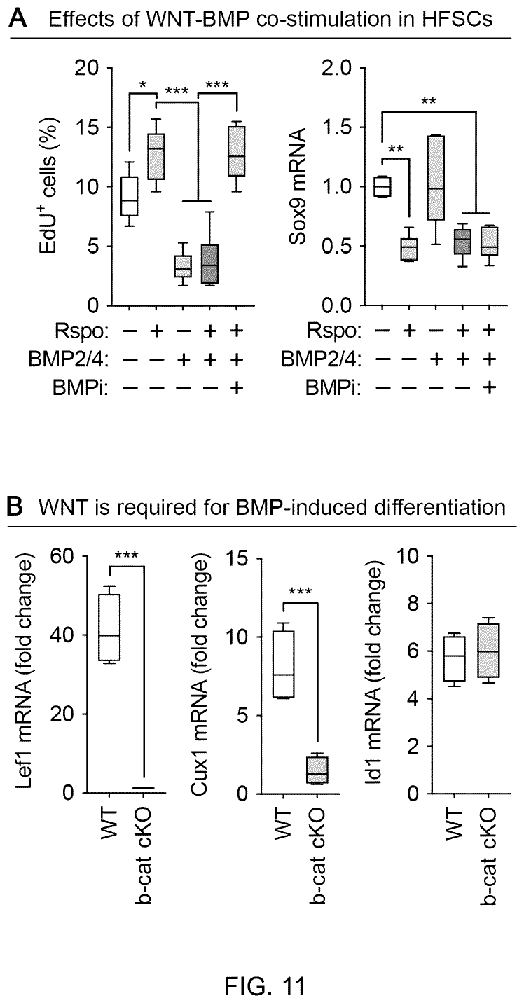

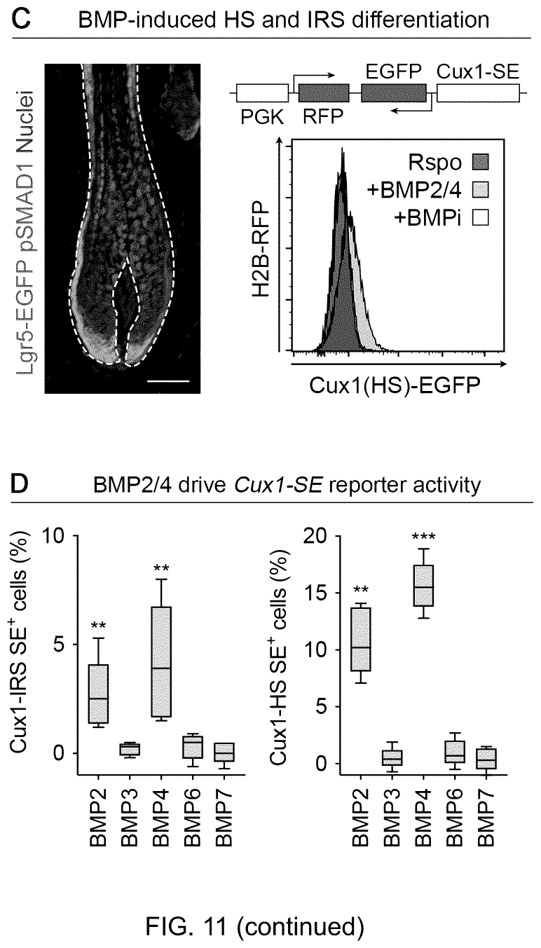

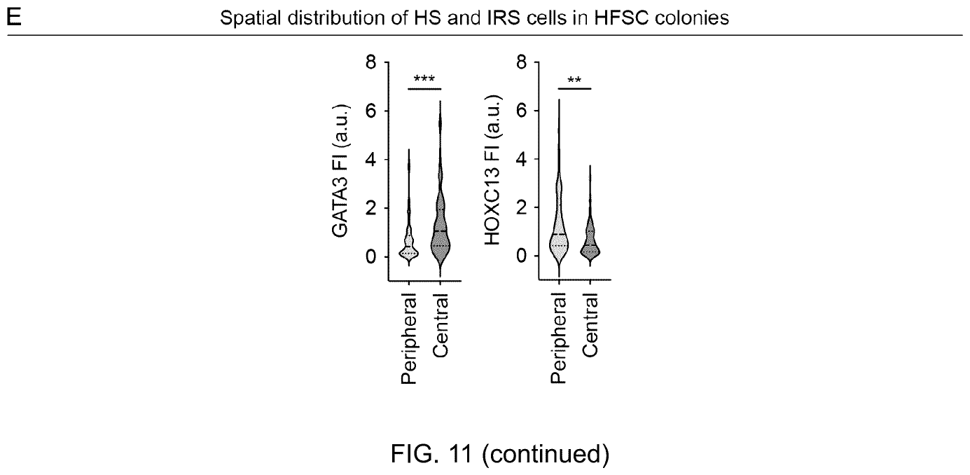

One question in the field of tissue stem cells has been that while Lgr5 and Lef1/Tcf1 are canonical WNT targets, their expression can sometimes be mutually exclusive, as they are in the HF (FIG. 2 Panel C). This becomes apparent as the hair cycle progresses, when WNT-active cells bifurcate into the expanding Lgr5⁺ ORS and more committed Lgr5^{neg}Lef1⁺ TACs that are marked by Cux1 (FIG. 3 Panel A). Using a described culture system, we dissected the underlying reasons. In contrast to Lef1/Tcf1, whose activation was independent of atRA, Lgr5-EGFP required both WNT activation and atRA for optimal activity (FIG. 10 Panel E). Additionally, although Lef1/Tcf1, Lgr5, and Cux1 depend upon WNT/Rspo, Lgr5 relied upon BMP inhibition while Cux1 expression was optimal under BMP^{hi} conditions, thereby bifurcating progenitors into the discrete lineages of the growing HF (FIG. 3 Panel B, FIG. 11, Panels A and B).

Employing a described culture system to disentangle the signals that distinguish HS and IRS differentiation, we took advantage of distinct Cux1-associated enhancer elements that faithfully drive reporter activity in these separate fates (FIG. 3 Panel C, FIG. 11 Panel C). Using these reporters, we discovered that similar to our findings on HFSC lineage induction (FIG. 2), so too was TAC lineage specification blocked under conditions that resodeed lineage plasticity (FIG. 3 Panel D). Resolving this condition enabled dual WNT and BMP activation to drive partial activity in the Cux1 (IRS) and Cux1 (HS) reporters, confirmed by lineage-specific transcription factor GATA3 and HOXC13 expression, respectively (FIG. 3 Panel E). BMP2 and BMP4 alongside Rspo were most effective in upregulating reporter activity (FIG. 11 Panel D). Within colonies where dual expression was observed, GATA3 was more central while HOXC13 was enriched in peripheral cells (FIG. 11 Panel E).

Probing for additional regulators that distinguish between the IRS and HS fates, we discovered that RA receptor inhibition (RARi) primarily increased HOXC13 expression and enhancer epicenter reporter activity for the HS lineage (FIG. 3 Panel E). To test whether RA activity similarly alters the balance between IRS and HS cells in vivo, we applied atRA or RARi to the back skin of mice at mid-anagen, when the HS and IRS begin to form (FIG. 3 Panel F). Topical atRA substantially reduced the size of the hair bulb overall and reduced the relative proportion of HOXC13⁺ to GATA3⁺ cells (FIG. 3 Panel E). These data provide evidence that formation of the hair shaft relies upon repression of RA activity.

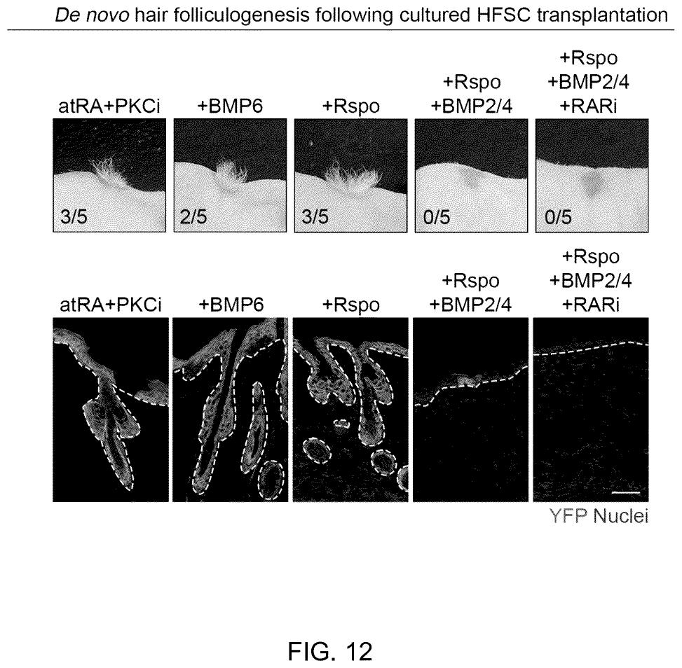

By overcoming the hurdles to lineage progression in vitro, we challenged the in vivo regenerative potential of the described lineage-specified cell populations. YFP-labeled progenitors from each group were combined with allogenic dermal fibroblasts and transplanted onto mouse back skins. Upon engraftment, the HFSCs efficiently produced hair when they had been precultured in serum-free media primed with either atRA, BMP6 or Rspo (FIG. 12). However, if HFSCs were co-treated with Rspo and BMPs, with or without RAR inhibitors, hair production was abolished. These data strongly suggested that cell populations that had advanced beyond the progenitor state in vitro were irreversibly committed differentiated lineage.

Overall, these findings underscored the value of the described system in dissecting complex signaling intersections that occur within bulge HFSCs as they transition from quiescence to active hair regeneration. By employing this system, we uncovered a previously under-appreciated importance of atRA and BMPs as critical modifiers of some but not all canonical WNT targets. These newfound insights resolved several previously perplexing observations that cut across WNT signaling in other tissues as well. Among findings revealed by the present disclosure was the importance of resolving HFSC lineage plasticity as a prerequisite to generating a niche microenvironment that is conducive to receiving the multifaceted signals required to define the identity of HFSCs and their discrete lineages.

### Example 5

Mapping Cultured HFSCs to the In Vivo HF with Single Cell Transcriptomics

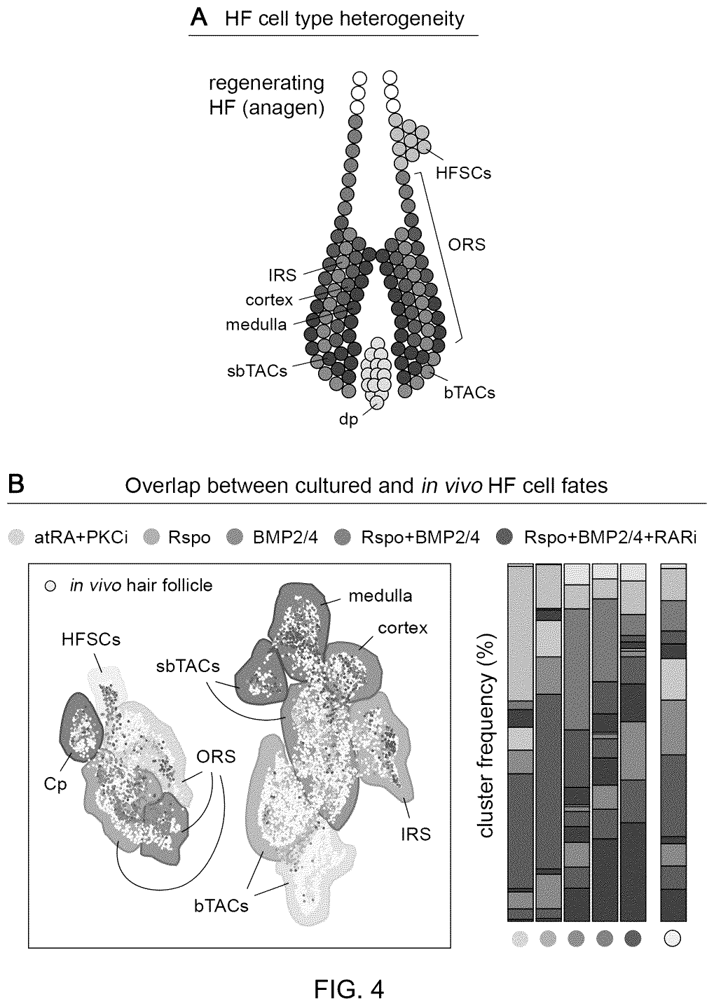

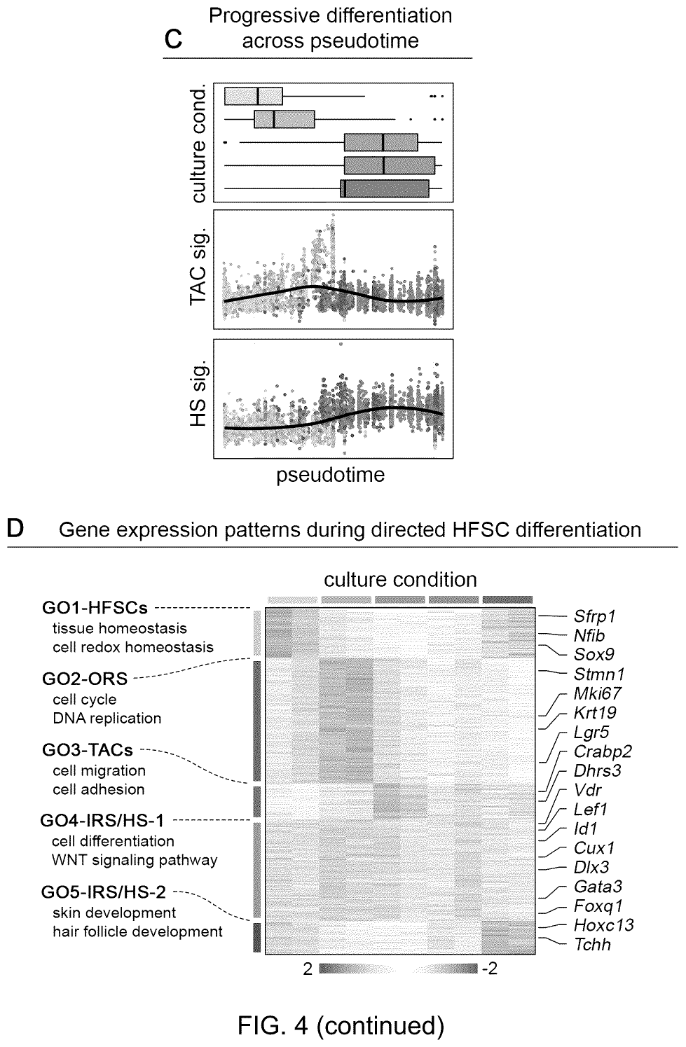

To evaluate the similarities between the HF differentiation program in vivo and that produced by the described stepwise differentiation program in vitro, we performed single cell RNA sequencing. Using transcriptomes of FACS-purified cells from autologous anagen-phase HFs as a reference dataset (FIG. 4 Panel A), we evaluated in-depth the characteristics of every culture-derived HF cell type (serum-free media containing optimal concentrations of atRA and PKCi, stimulated with Rspo, BMPs, and RARi) after their integration with Harmony.

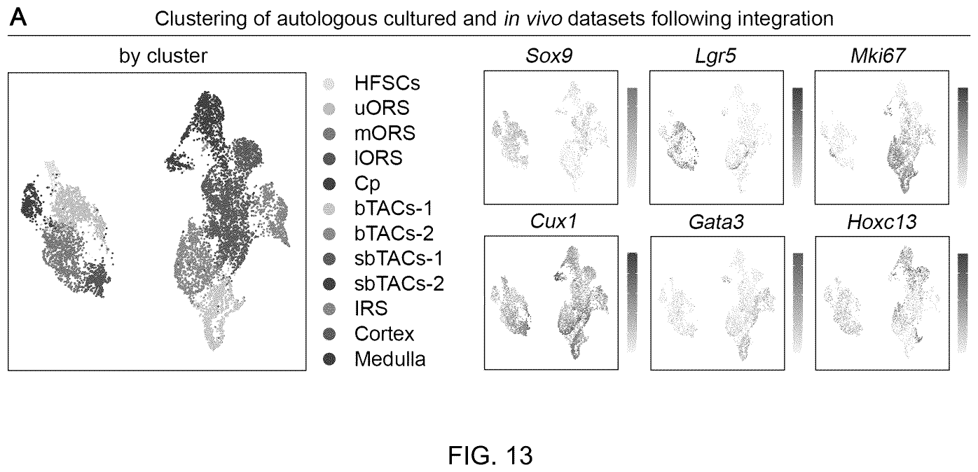

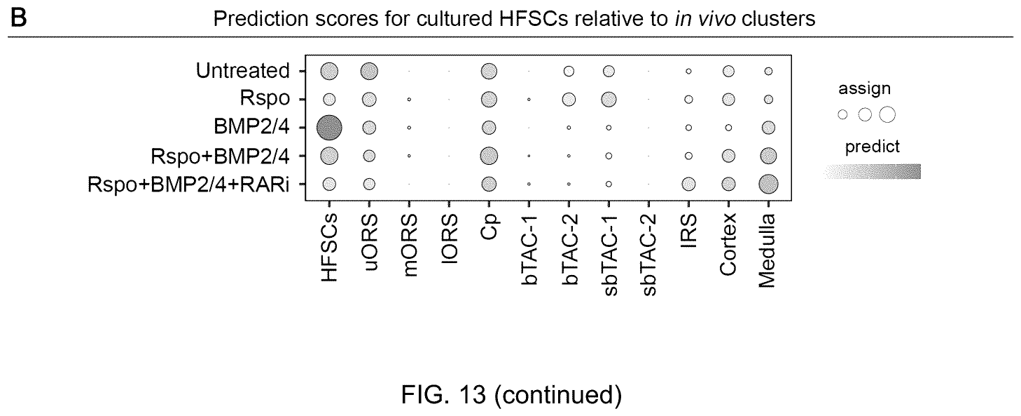

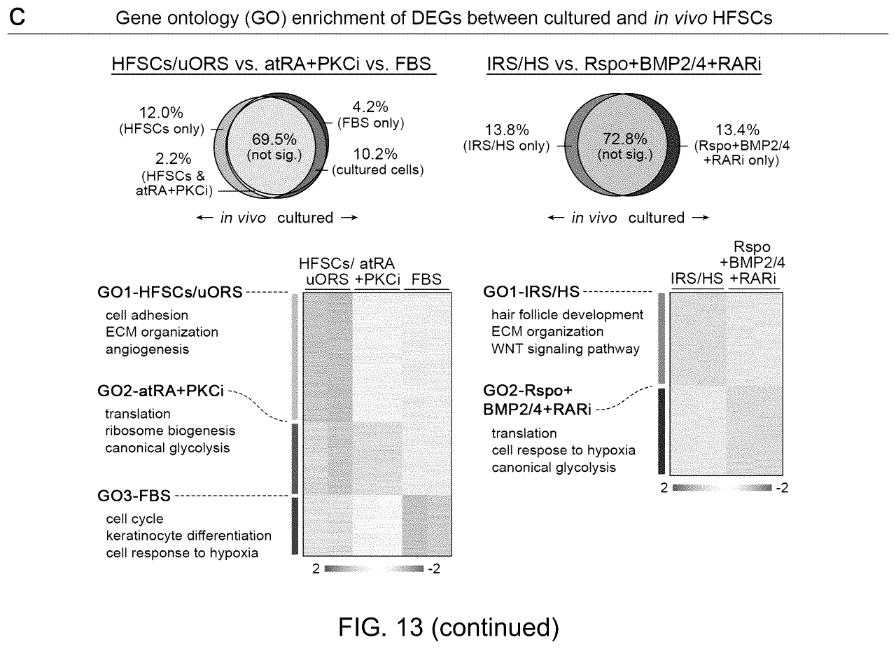

Clustering following dimensional reduction by ‘uniform manifold approximation and projection’ (UMAP) identified 12 annotated cell types that captured the full array of HF fates (FIG. 4 Panel B, FIG. 13 Panel A). Similar results were achieved using cluster assignments and prediction scores calculated with a weighted nearest neighbor approach (FIG. 13 Panel B). Cultured HFSCs exposed to atRA and PKCi predominantly clustered with the upper ORS (uORS), known to retain stemness across hair cycles, and showed greater similarity to the bulge and uORS than HFSCs grown in serum-containing media (FIG. 13 Panel C). HFSCs exposed to Rspo were most analogous to shorter lived TACs, while HFSCs treated with Rspo and BMPs corresponded to the major lineages of mature HFs, including IRS, hair shaft (both medulla and hair cortex).

As judged by Monocle3 to infer the trajectory of HFSC differentiation, SOX9⁺ HFSCs followed an orderly route by advancing through a TAC-like state and terminating in the robust induction of hair shaft genes (FIG. 4 Panel C). BMPs drove positioning along this axis, advancing cells to later stages of pseudotime. Differential gene expression (DEG) identified ‘signature genes’ and biological processes within each HF cell type, corroborating the fidelity of the described directed differentiation protocols. A high degree of similarity was also observed for the comparison of dual WNT/BMP-activated HFSCs with differentiated IRS and HS clusters. Overall, the ability to define conditions where lineage specification and differentiation of HFSCs can be achieved outside the context of their native niches suggests that soluble signals are central to lineage specification.

Seeking potential mechanisms underlying stem cell fate decisions, we performed pathway enrichment analysis and identified spontaneous epidermal differentiation among the most significant features prevented by atRA and PKCi (FIG. 13 Panel C). Of additional note and regardless of cell fate, ECM assembly, global translation, and glycolysis were enriched in cultured stem cells compared to their in vivo counterparts (FIG. 13 Panel C). Consistently, while bulge HFSCs in vivo form their own ECM, maintain low rates of translation, and rewire their metabolic networks, self-renewing HFSCs in vitro must overcome these constraints. Altogether, these data again underscored the power of suppressing lineage plasticity as a prerequisite to maintain HFSC identity and lineage progression.

### Example 6

### RA Functions in Wound Repair by Regulating the Dynamics of Lineage Plasticity

When skin encounters a shallow wound, its epidermis and upper HF are denuded, leaving underlying bulge HFSCs to re-epithelialize these tissues in a fashion that avoids complications from wound edge effects, contraction, and scarring. Because wound-induced HFSCs first enter a transient state of lineage plasticity prior to making their fate switch to the epidermis, our data thus far illuminated atRA as a possible key candidate for regulating the balance between homeostasis and wound repair.

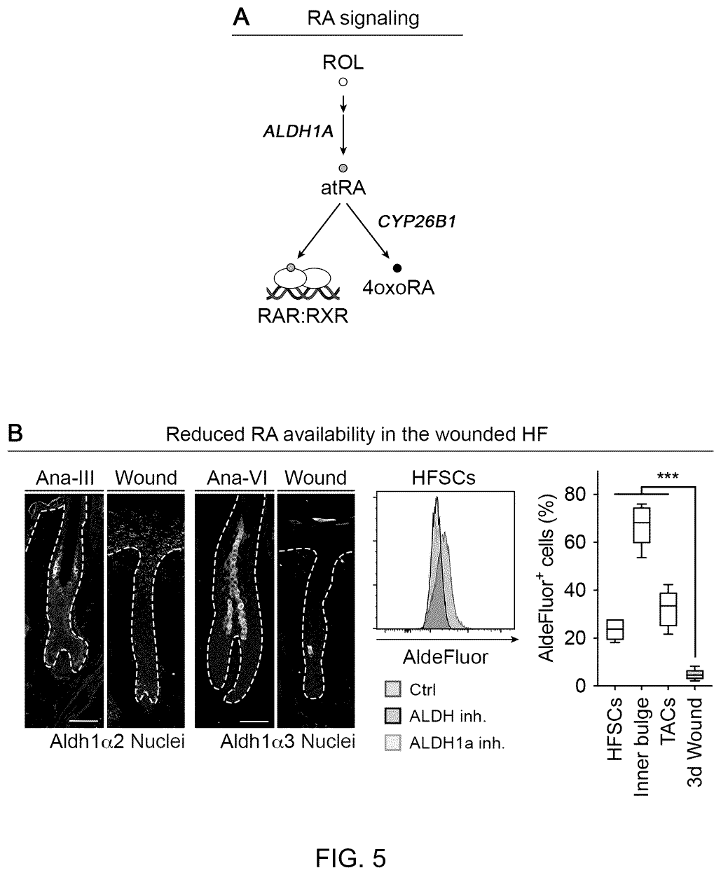

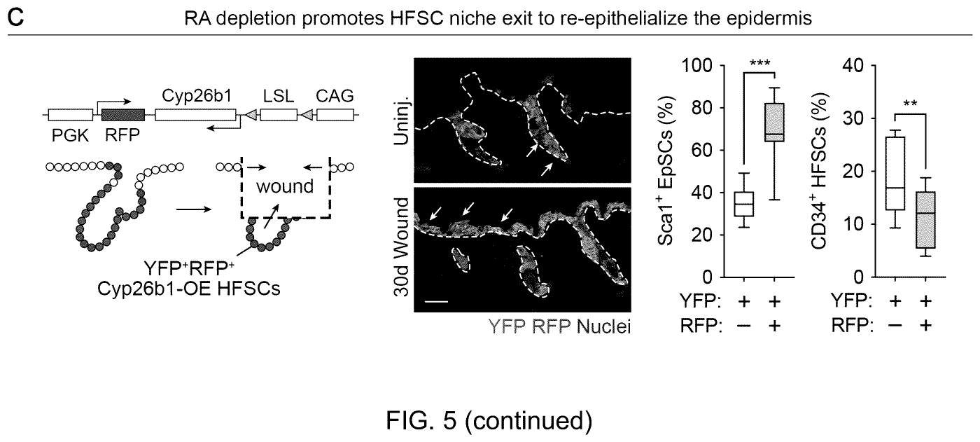

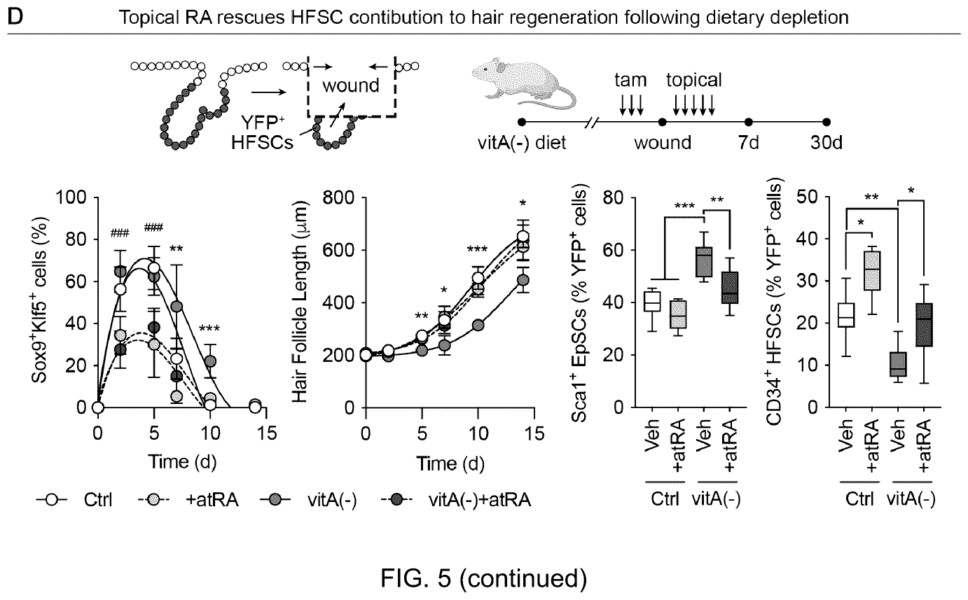

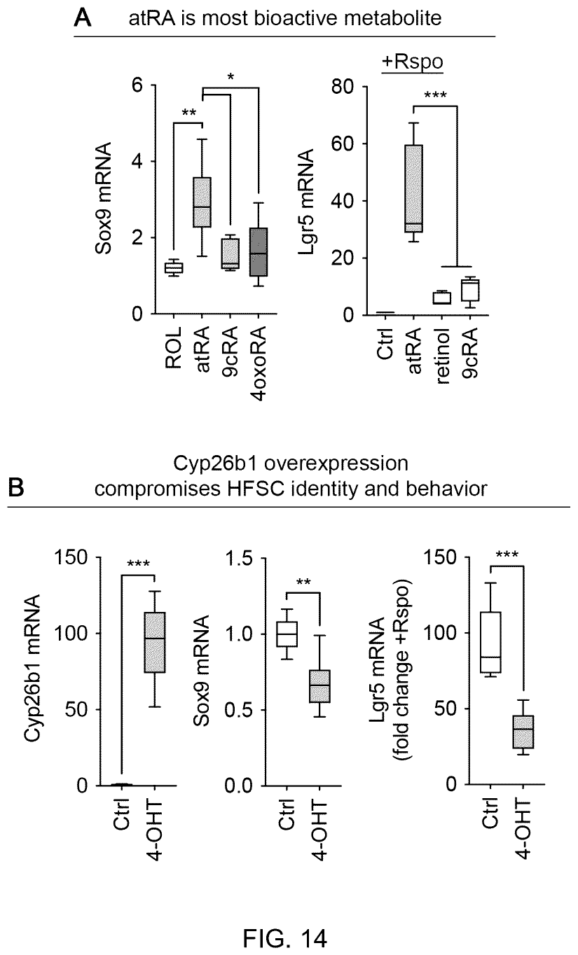

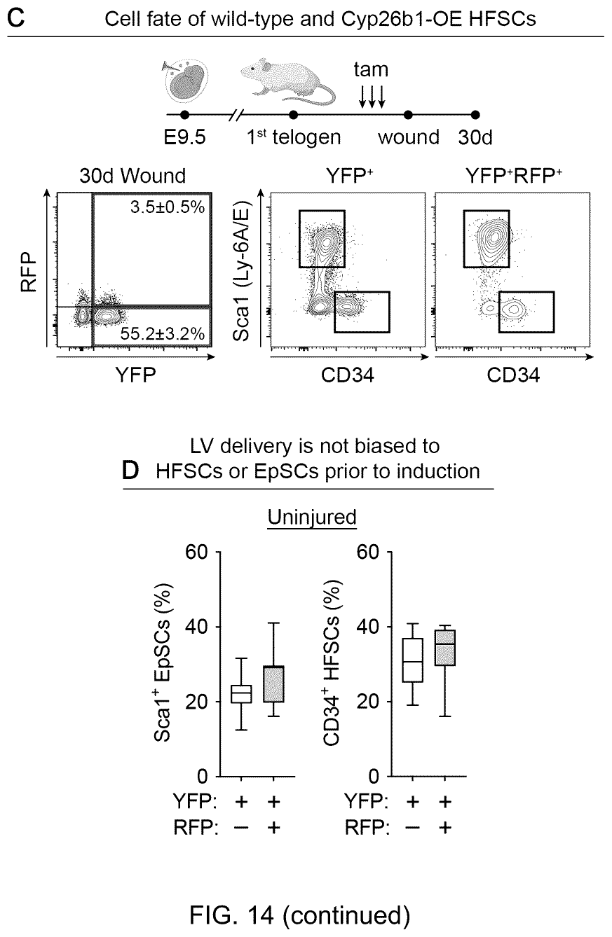

As atRA is synthesized locally in skin from circulating retinol (FIG. 5 Panel A), we first addressed whether the bulge niche is a site of atRA production and whether it might be affected by injury. Immunofluorescence and enzymatic activity assays localized rate-limiting retinaldehyde dehydrogenases (ALDH1A) to the inner bulge layer adjacent to quiescent HFSCs as well as within the cortical layer of the hair shaft (FIG. 5 Panel B). Correspondingly, among the various physiological vitamin A derivatives, atRA exerted the greatest effect on Sox9 and Rspo-mediated Lgr5 upregulation in cultured HFSCs (FIG. 14 Panel A). Notably, ALDH1A activity as a proxy for atRA levels plummeted following injury and then returned during the healing process (FIG. 5 Panel B).

Given these results, we analyzed whether the reduction in local atRA in the bulge niche may be directly responsible for mobilizing HFSCs to re-epithelialize the missing epidermis. To test this, we first intrinsically manipulated retinoid activity within HFSCs without perturbing the natural temporal changes of atRA that occur in wounded skin. To do so, we used our in utero delivery system to mosaically transduce the skin epithelium of Sox9CreER;Rosa26-LSL-YFP mice with a lentivirus encoding an RFP marker and a constitutive Cyp26b1, encoding a monooxygenase that catalyzes the degradation of atRA to its relatively inactive form, 4-oxo-RA (FIG. 5 Panel A and FIG. 14 Panel B). Analyses of the wound responses of YFP-lineage traced HFSCs suggested that RFP⁺Cyp26b1⁺ HFSCs were significantly more likely than their WT counterparts in contributing to epidermal repair, at the expense of remaining within the HF ‘bulge’ (FIG. 5 Panel C, FIG. 14, Panels C and D).

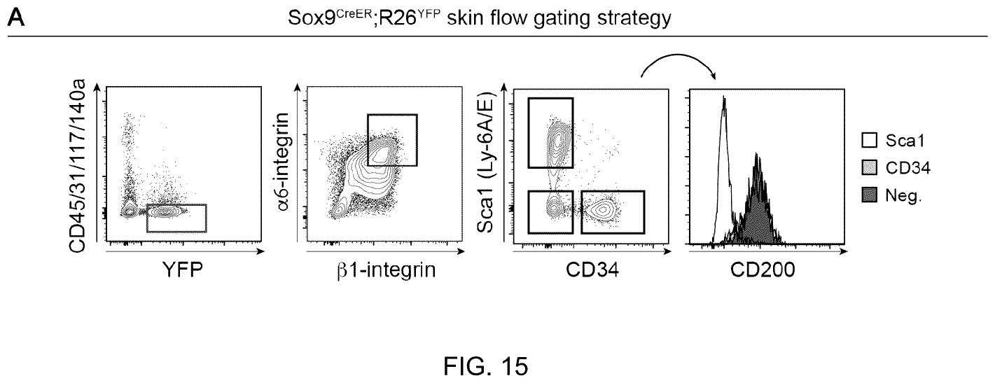

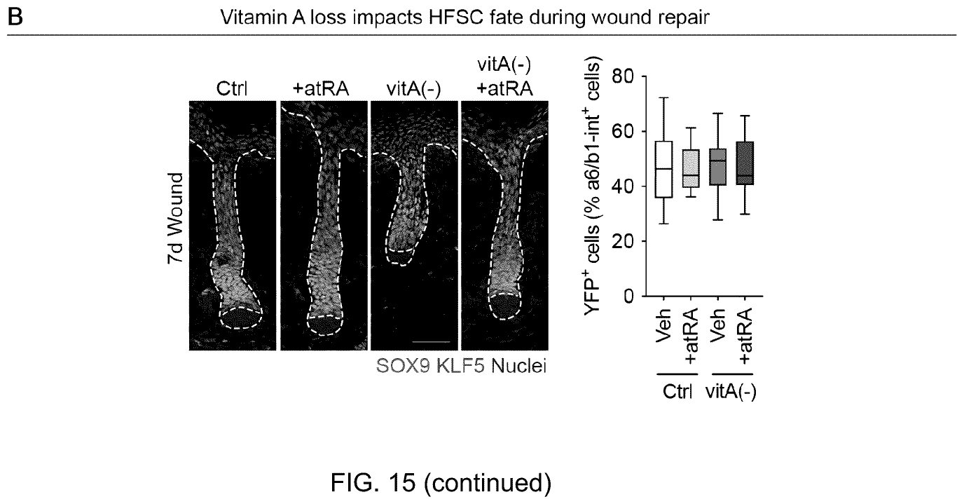

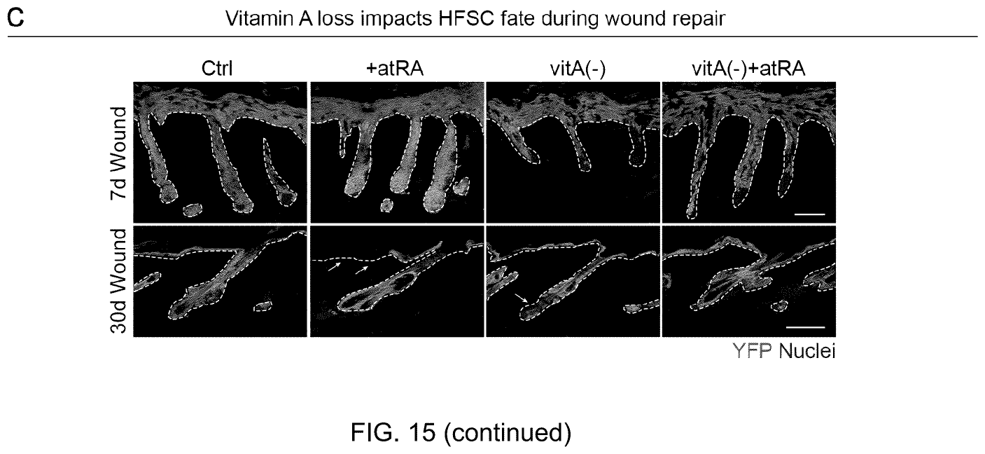

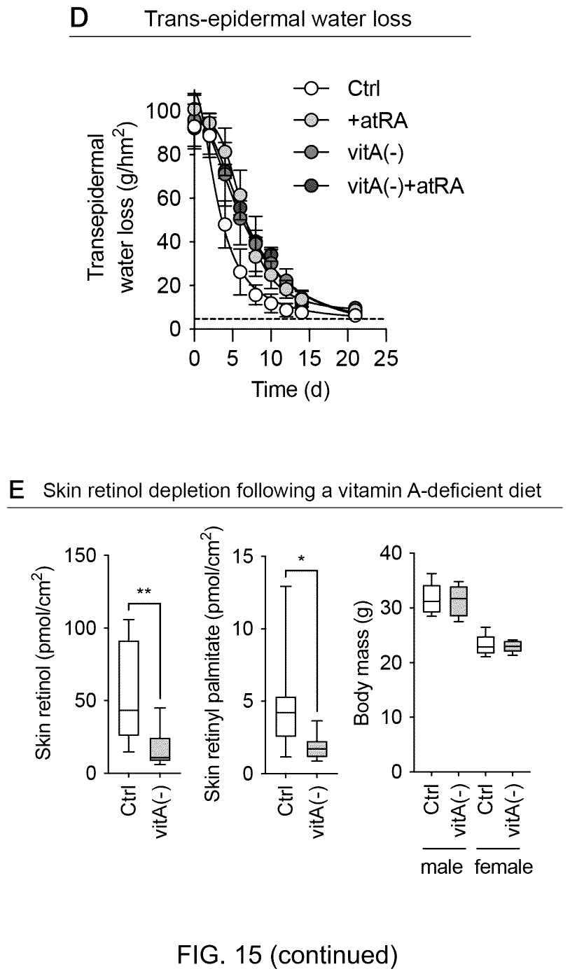

The recruitment of HFSCs to epidermal repair is a multistep process, involving first the entry of HFSCs into a lineage plasticity state, second the fate switch to an epidermal fate and then later, the rebalancing of stem cell numbers across tissue compartments. Given the need for atRA to drive hair lineage determination, we analyzed whether elevating atRA after injury might diminish the ability of HFSCs to enter a state of lineage plasticity after wounding. To test this, we administered topical atRA immediately after wounding to telogen-phase, lineage marked HFSCs. While entry into the hair cycle proceeded normally after injury, topical atRA led to a stem cell imbalance (FIG. 5 Panel D). Fewer HFSCs entered the lineage plasticity state during the early stages of repair, resulting in a higher fraction of HFSCs remaining in the bulge instead of undergoing a fate change to become EpSCs (FIG. 5 Panel D; FIG. 15, Panels A to C). The consequences of diminishing lineage infidelity pools were substantial, as atRA caused a clear delay in restoring the skin's barrier following injury when judged by transepidermal water loss (TEWL) (FIG. 15 Panel D).

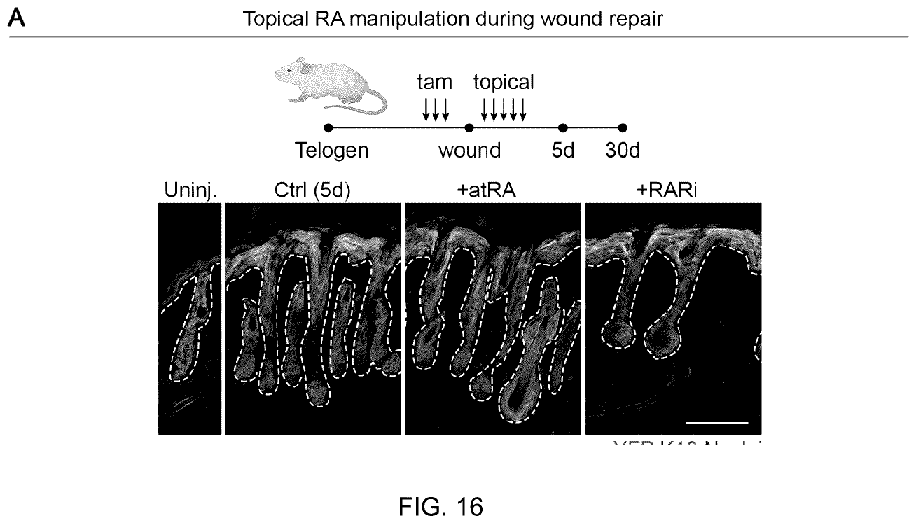

To assess the consequences of promoting lineage plasticity following injury, we placed mice on a vitamin A-deficient diet that depleted the skin of retinol without affecting body mass (FIG. 15 Panel E). In this case, following tissue injury, mobilized HFSCs entered a prolonged stated of dual lineage potential well into the later stages of repair (FIG. 5 Panel D). In striking contrast to atRA excess, the HFs of wounded vitamin A deficient mice exhibited a marked reduction in hair regeneration relative to their WT counterparts (FIG. 5 Panel D). By day 30, vitamin A deficiency also elevated the fraction of stem cells that contributed to the repaired epidermis and diminished the number remaining in the bulge. Similar results were obtained with topical RAR inhibitor (RARi), reducing HF downgrowth and promoting epidermal upward flux following wounding (FIG. 16). Moreover, all of these features were rescued by topically applying atRA to the wound bed of vitamin A-deficient mice, underscoring the tissue-specific nature of the response (FIG. 5 Panel D).

Despite the increased flux into lineage plasticity, restoration of the skin barrier was delayed in the face of vitamin A deficiency (FIG. 15 Panel D). In this case, however, the perturbation appeared to be rooted in a delay in resolving lineage infidelity and restoring homeostasis, rather than an impediment in activating HFSCs and fueling their entry into the lineage infidelity state.

### Example 7

The Example extends the foregoing Examples and provides a description of media testing and improvements, and provides a comparison of an initial and improved media in Table B.

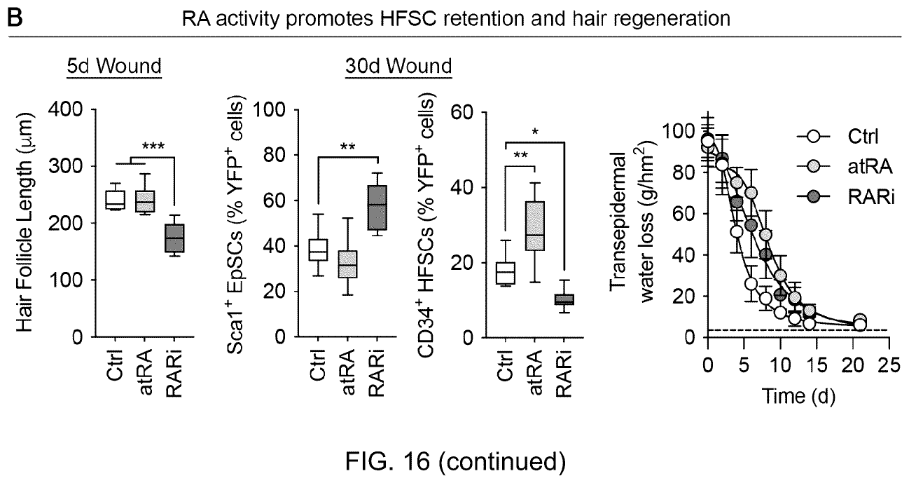

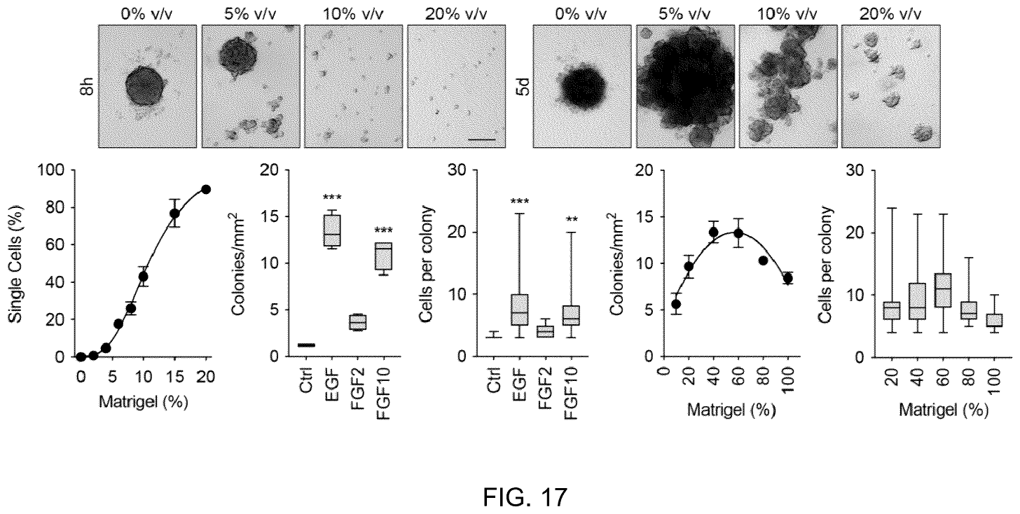

Initial experiments were conducted in standard HFSC media and first aimed to establish conditions for culturing HFSCs in 3D, encapsulated in Matrigel under low-attachment, hydrophobic conditions. Other ECM components, including collagen I and fibronectin, resulted in spread morphologies that do not recapitulate in vivo HFSC biology and impeded later attempts to resolve lineage plasticity. HFSCs formed aggregates with relatively low Matrigel amounts (diluted in standard media), remaining uniformly dispersed as single cells only at concentrations >20% v/v. HFSCs required an added mitogen and >5% Matrigel to proliferate in 3D. Maximal rates of cell proliferation were reached with 25 ng/ml epidermal growth factor (EGF) or 10 ng/ml fibroblast growth factor 10 (FGF10). Combining these mitogens together had no additive effect. Colony forming efficiency (CFE) was optimal in a range of 40-60% Matrigel and the total number of cells within each colony after 7 days was greatest at 60%. Therefore, we moved forward with 60% Matrigel to best allow for HFSC expansion as single cell colonies in 3D, as shown in FIG. 17.

Some base media formulations remain unmodified from the standard media composition, including the ratio DMEM to F12 basal media (3:1), sodium bicarbonate (pH), or penicillin/streptomycin (antibiotics). A range of CaCl₂) (0.1-1.5 mM) was tested but was not changed from the standard media formulation (0.3 mM), a Less CaCl₂ reduced cell proliferation while higher amounts promoted epidermal differentiation. Y-27632 (ROCK inhibitor) was kept for its ability to increase cell survival and CFE upon isolation. Hydrocortisone and cholera toxin (cAMP modulator) were removed from the media, as they were not necessary for colony formation or cell proliferation in 3D cultures.

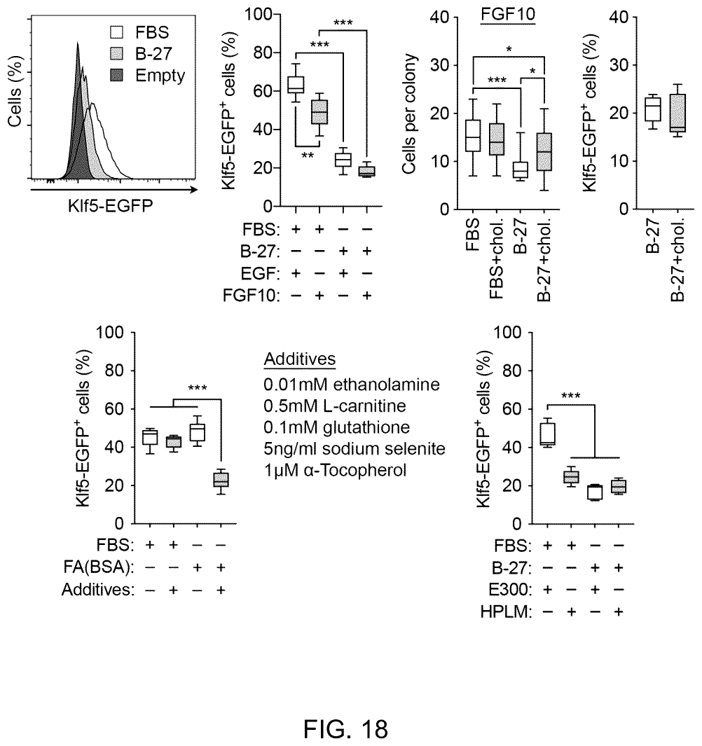

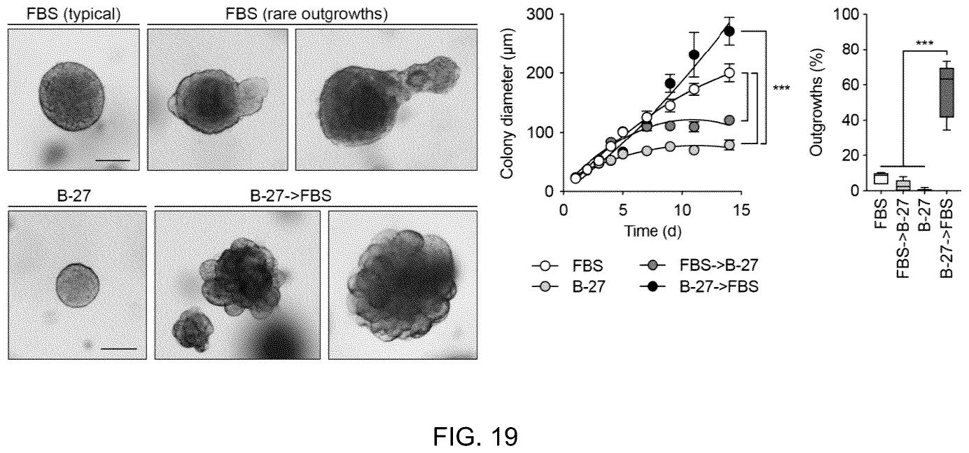

Once base conditions were initially established for HFSC growth and survival in 3D cultures, similar modifications were screened for their ability to reduce lineage plasticity as measured by a Klf5-EGFP transgene. Replacing fetal bovine serum (15% FBS) with a commercially available serum-free media supplement (B-27; Thermo Fisher) partially reduced Klf5-EGFP levels without compromising colony forming efficiency. Klf5-EGFP levels were also consistently lower in the presence of FGF10 rather than EGF. Cholesterol (solubilized using methyl-β-cyclodextrin) was added to serum-free media to increase colony growth without compromising reductions in lineage plasticity. Increased B-27 had no effect on colony forming efficiency, HFSC proliferation, or Klf5-EGFP levels. N-2 supplement was also tested in place of B-27 but did not have the same effect. Serum replacement and a series of culture additives (primarily antioxidants) were both required to reduce lineage plasticity, as neither replacement with a cocktail of fatty acids (0.025 mM palmitate and 100 mg/ml oleate/linoleate conjugated to bovine serum albumin) and solubilized cholesterol or the addition of these additives in the presence of serum alone reduced Klf5-EGFP levels. Each of these additives were required to achieve a complete rescue, although glutathione and sodium selenite had the greatest impact when removed. A range of fatty acid concentrations (0.01-05 mM palmitate and 10-250 mg/ml oleate/linoleate) were tested, with HFSC colony size plateauing at the chosen concentrations. The inclusion of all three was optimal for HFSC growth, although palmitate was able to sustain a reduced level of HFSC growth alone. Media formulated to mimic human plasma (Gibco) reduced Klf5-EGFP levels even in the presence of serum and could not be further lowered using the same pool of culture additives, as shown in FIG. 18.

HFSC growth patterns and overall colony structure were also affected by the presence of serum. Rare outgrowths from the typically rounded colony shape occurred with serum and did not occur after its removal. Toggling between serum and serum-free conditions showed this phenotype to be reversible, as serum removal stabilized previously unchecked colony growth while its reintroduction resulted in the formation of many distinct outgrowths originating in a single colony. These effects were independent of the amount of serum present in these cultures (5-15%), although cell proliferation was reduced as serum volume was lowered. Results are shown in Figure C.

To further reduce lineage plasticity, we screened a library of small molecules that have been broadly implicated in wounding. Of the >70 compounds tested, competitive inhibitors of extracellular signal-regulated kinase 1/2 (ERK1/2), the calcium-dependent protein kinase C isoforms a/b (PKCi), and all-trans-retinoic acid (atRA) proved effective at reducing lineage plasticity without affecting proliferation in cultured HFSCs. The library is shown in Table A.

TABLE A

| Small molecule/growth factor | Solvent | Working conc. | |
|---|---|---|---|
| 10058-F4 | DMSO | 0.025 | mM |
| 17-AAG | DMSO | 0.002 | mM |
| 9-cis-RA | DMSO | 0.001 | mM |
| A83-01 | DMSO | 0.001 | mM |
| abrocitinib | DMSO | 0.01 | mM |
| AG490 | DMSO | 0.01 | mM |
| AG879 | DMSO | 0.01 | mM |
| AICAR | PBS | 1 | mM |
| Ascorbate | dH2O | 0.025 | mM |
| atRA | DMSO | 0.001 | mM |
| b-estradiol | DMSO | 0.001 | mM |
| BMP2 | 0.1% BSA in PBS | 0.02 | ug/ml |
| BMP3 | 0.1% BSA in PBS | 0.02 | ug/ml |
| BMP4 | 0.1% BSA in PBS | 0.005 | ug/ml |
| BMP6 | 0.1% BSA in PBS | 0.02 | ug/ml |
| BMP7 | 0.1% BSA in PBS | 0.025 | ug/ml |
| Calcipotriol | DMSO | 0.00001 | mM |
| Calcitriol | DMSO | 0.00001 | mM |
| CGP77675 | DMSO | 0.001 | mM |
| CH223191 | DMSO | 0.001 | mM |
| CHIR99021 | DMSO | 0.001 | mM |
| CID 5951923 | DMSO | 0.01 | mM |
| CP775146 | DMSO | 0.001 | mM |
| DAPT | DMSO | 0.01 | mM |
| dmPGE2 | Methylacetate | 0.001 | mM |
| EDA-A1 | 0.1% BSA in PBS | 0.25 | ug/ml |
| EGF | 0.1% BSA in PBS | 0.05 | ug/ml |
| fedratinib | DMSO | 0.01 | mM |
| FGF10 | 0.1% BSA in PBS | 0.01 | ug/ml |
| FGF18 | 0.1% BSA in PBS | 0.05 | ug/ml |
| Forskolin | DMSO | 0.01 | mM |
| FR180204 | DMSO | 0.01 | mM |
| Gefitinib | DMSO | 0.01 | mM |
| Gö6976 | DMSO | 0.001 | mM |
| Gö6983 | DMSO | 0.005 | mM |
| GSK0660 | DMSO | 0.001 | mM |
| GW501516 | DMSO | 0.001 | mM |
| GW6471 | DMSO | 0.001 | mM |
| GW9662 | DMSO | 0.001 | mM |
| H89 | DMSO | 0.005 | mM |
| ICG-001 | DMSO | 0.005 | mM |
| Ionomycin | DMSO | 0.001 | mM |
| IQ-1 | DMSO | 0.005 | mM |
| JNK-IN-8 | DMSO | 0.001 | mM |
| LDN193189 | DMSO | 0.00005 | mM |
| LG100268 | DMSO | 0.001 | mM |
| LY294002 | DMSO | 0.01 | mM |
| M1 Fusion | DMSO | 0.02 | mM |
| Metformin | PBS | 0.001 | mM |
| ML264 | DMSO | 0.01 | mM |
| OSM | 0.1% BSA in PBS | 0.1 | ug/ml |
| PD0325901 | DMSO | 0.001 | mM |
| Pluripotin | DMSO | 0.001 | mM |
| PMA | DMSO | 0.00005 | mM |
| Prolactin | 0.1% BSA in PBS | 0.5 | ug/ml |
| Prostratin | DMSO | 0.001 | mM |
| ritlecitinib | DMSO | 0.01 | mM |
| Rspo1 | 0.1% BSA in PBS | 0.25 | ug/ml |
| Rspo2 | 0.1% BSA in PBS | 0.2 | ug/ml |
| Rspo3 | 0.1% BSA in PBS | 0.2 | ug/ml |
| ruxolitinib | DMSO | 0.01 | mM |
| SAG | DMSO | 0.0001 | mM |
| SB202190 | DMSO | 0.01 | mM |
| SB203508 | DMSO | 0.01 | mM |
| SB431542 | DMSO | 0.001 | mM |
| SB590885 | DMSO | 0.001 | mM |
| SHH | 0.1% BSA in PBS | 0.1 | ug/ml |
| Somatostatin | PBS | 0.00005 | mM |
| SP100030 | DMSO | 0.005 | mM |
| SP600125 | DMSO | 0.02 | mM |
| SU5402 | DMSO | 0.01 | mM |
| T5524 | DMSO | 0.01 | mM |
| TGFb | 0.1% BSA in PBS | 0.005 | ug/ml |
| TK216 | DMSO | 0.01 | mM |
| tofacitinib | DMSO | 0.01 | mM |
| TUDC | EtOH | 0.05 | mM |
| VER115008 | DMSO | 0.005 | mM |
| Verteporfin | DMSO | 0.001 | mM |
| WH-4-023 | DMSO | 0.001 | mM |
| Wnt-C59 | DMSO | 0.00001 | mM |
| Wnt3a | 0.1% BSA in PBS | 0.1 | ug/ml |
| Wnt5a | 0.1% BSA in PBS | 0.1 | ug/ml |
| Wnt7a | 0.1% BSA in PBS | 0.25 | ug/ml |
| Wortmannin | DMSO | 0.005 | mM |
| XAV939 | DMSO | 0.001 | mM |
| Yhhu3792 | DMSO | 0.001 | mM |
| YK-4-279 | DMSO | 0.01 | mM |

The structures of all the compounds in Table A are known.

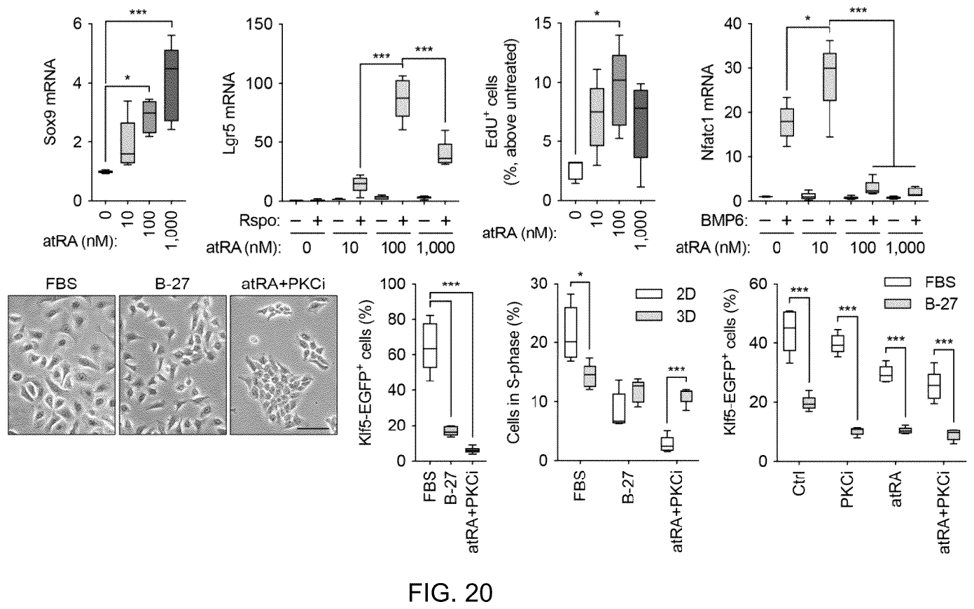

After testing each in combination, atRA and PKCi proved most effective at reducing Klf5-EGFP levels. ERKi was also excluded because of its known ability to regulate the initiation of hair growth. Both atRA (10 nM-1 μM) and PKCi (0.1-10 μM) were titrated to determine the optimal concentration to reduce lineage plasticity and promote physiological HFSC behavior. 1 μM PKCi was selected as the minimum concentration required to achieve its maximal effect on lineage plasticity and HFSC fate. atRA, on the other hand, demonstrated dose-dependent effects on markers of basal HFSC identity and their responsiveness to niche growth factors that regulate HFSC behavior, in this case BMP6 (promotes HFSC quiescence during the resting phase of the hair cycle) and R-spondin (Rspo; promotes stem cell activation and hair cycling during anagen entry). Although atRA increased Sox9 mRNA levels in a dose-dependent manner, Lgr5 mRNA upregulation (activated stem cell marker downstream of WNT activation) and EdU incorporation were optimized at 100 nM atRA. In contrast, BMP6-mediated Nfatc1 mRNA upregulation (quiescence marker) was maximal at 10 nM. Therefore, the amount of atRA should be modified in HFSC cultures to support the desired cell fate in combination with these and possibly other growth factors. Finally, the 3D environment was critical for HFSC growth in these conditions, as a combination of serum removal and the addition of atRA and PKCi were able to suppress lineage plasticity but did not support proliferation or long-term cell survival. Results are shown in FIG. 20.

Based in part on this Example and the foregoing Examples, the disclosure provides the following Table B, which provides a comparison of standard media components to new media components. In an example is the media used to generate hair follicle stem cells comprises one or more components listed under “New” in Table B, including but not limited at the stated concentrations, and includes excluding any component where an amount is not listed:

TABLE B

| | | This | |
|---|---|---|---|
| Media Component | Standard | disclosure | |
| DMEM/F12(3:1) | 12.5 | 12.5 | g/l |
| CaCl2 | 0.3 | 0.3 | mM |
| Y-27632 | 0.01 | 0.01 | mM |
| Sodium bicarbonate | 3.07 | 3.07 | g/l |
| L-alanyl-L-glutamine (GlutaMax) | 217.2 | — | mg/l |
| FBS | 15 | — | % |
| hydrocortisone | 0.4185 | — | mg/l |
| cholera toxin | 0.0094 | — | mg/l |
| EGF | 0.05 | — | mg/l |
| B-27 supplement (without vitamin A)* | — | 1x | — |
| cholesterol-cyclodextrin | — | 5 | mg/l |
| ethanolamine | — | 0.01 | mM |
| L-glutamine | — | 474.5 | mg/l |
| all-trans retinoic acid | — | 0.0001 | mM |
| Go-6976 | — | 0.001 | mM |
| FGF10 | — | 0.01 | mg/l |

| *B-27 supplement can be replaced with the following: | | |
|---|---|---|
| Palmitate (BSA-conjugated) | 0.025 | mM |
| Oleate (BSA-conjugated) | 100 | g/l |
| Linoleate (BSA-conjugated) | 100 | g/l |
| DL-a-tocopherol acetate | 0.001 | mM |
| L-carnitine | 0.5 | mM |
| Sodium selenite | 5 | ng/ml |
| Glutathione | 0.1 | mM |
| T3 | 0.0001 | uM |
| Apo-transferrin | 5.22 | ug/ml |
| Insulin | 0.5 | ug/ml |

### Example 8

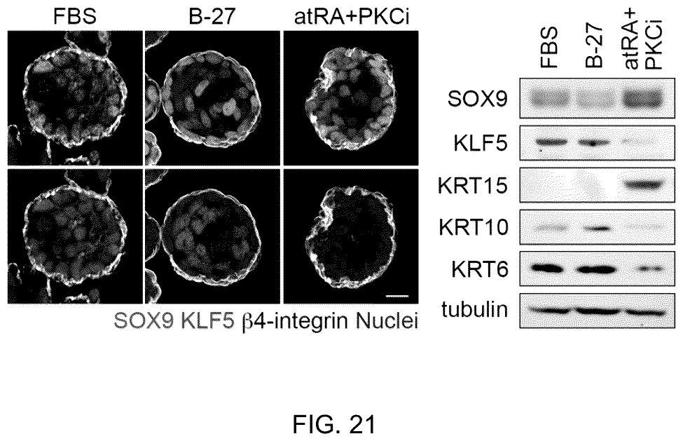

This example relates to FIG. 21. With respect to FIG. 21, to assess the translatability of the described methodology enabling the faithful recapitulation of hair follicle stem cell (HFSC) identity in culture to humans, we introduced the described conditions developed in the mouse to human keratinocytes. After 7 days in culture, replacement of fetal bovine serum (FBS) with a serum-free media supplement (B-27) reliably limited KLF5-expressing cells to the inward-stratifying suprabasal layers of clonal colonies and increased the expression of KRT10, a marker of epidermal differentiation. The addition of all-trans retinoic acid (atRA) and the calcium-dependent protein kinase C inhibitor (PKCi) G66976 further suppressed KLF5 expression in both basal and suprabasal layers and upregulated SOX9 levels, consistent with the response originally identified in murine stem cells. This was also evidenced by alterations in the keratin profile, including reductions in KRT10 and KRT6 (indicative of hyperproliferative epidermis in wounded or cancerous settings) and upregulation of KRT15, a known HFSC marker. Together, these results demonstrate the ability of these conditions to suppress the signatures of wound- and cancer-associated lineage plasticity and promote the acquisition of a HFSC fate using human cells.

While the disclosure has been described through specific examples, routine modifications will be apparent to those skilled in the art and such modifications are intended to be within the scope of the present disclosure.

What is claimed is:

1. An in vitro method of generating hair follicle stem cells, the method comprising: culturing epithelial cells in a cell culture medium comprising all-trans-retinoic acid (atRA) and a calcium-dependent protein kinase C inhibitor (PKCi) to thereby promote the epithelial cells to become the hair follicle stem cells.

2. The method of claim 1, wherein the PKCi specifically inhibits PKC calcium-dependent alpha and beta isoenzymes.

3. The method of claim 2, wherein the PKCi is Go-6976.

4. The method of claim 3, wherein the cell culture medium further comprises one or more of:
DMEM/F12
Calcium Chloride
Y-27632
Sodium bicarbonate;
cholesterol-cyclodextrin;
L-glutamine; and
a mitogen that is optionally fibroblast growth factor 10 (FGF10) or epidermal growth factor (EGF).

5. The method of claim 4, wherein the cell culture medium further comprises B-27 supplement without vitamin A; or a mixture comprising
Palmitate (BSA-conjugated)
Oleate (BSA-conjugated)
Linoleate (BSA-conjugated)
DL-a-tocopherol acetate
L-carnitine
Sodium selenite
Glutathione
T3
Apo-transferrin, and
insulin.

6. An isolated population of hair follicle stem cells made by the method of claim 3.

7. A method comprising exposing a test compound to the isolated population of hair follicle stem cells of claim 6, and determining whether or not the test compound influences a property of the hair follicle stem cells.

8. The method of claim 7, wherein the property is selected from survival of the hair follicle stem cells, or differentiation of the hair follicle stem cells.

9. A method comprising culturing the isolated hair follicle stem cells of claim 3 in serum-free media primed with one of BMP6 or R-spondin.

10. The method of claim 9, comprising mixing the isolated hair follicle stem cells with dermal fibroblasts.

11. The method of claim 10, comprising engrafting the isolated hair follicle stem cells and dermal fibroblasts onto skin of an individual to thereby generate hair.

12. A cell culture medium for use with hair follicle stem cells, the cell culture media comprising all-trans-retinoic acid (atRA) and a calcium-dependent protein kinase C inhibitor (PKCi).

13. The cell culture media of claim 12, further comprising:
DMEM/F12
Calcium Chloride
Y-27632
Sodium bicarbonate
cholesterol-cyclodextrin;
L-glutamine; and
and a mitogen that is optionally FGF10.

14. The cell culture medium of claim 12, wherein the cell culture medium further comprises B-27 supplement without vitamin A;
or a mixture comprising:
Palmitate (BSA-conjugated)
Oleate (BSA-conjugated)
Linoleate (BSA-conjugated)
DL-a-tocopherol acetate
L-carnitine
Sodium selenite
Glutathione
T3
Apo-transferrin, and
Insulin.

15. The cell culture of claim 14, comprising the mixture.
